(19) United States
(12) Patent Application Publication
(10) Pub. No.: US 2023/0250410 A1
(43) Pub. Date: Aug. 10, 2023

(54) RECOMBINANT ACE2-FC FUSION MOLECULES AND METHODS OF MAKING AND USING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Tsung-I TSAI, Redmond, WA (US); Nga Sze Amanda MAK, Mukilteo, WA (US); Andrew WAIGHT, Kenmore, WA (US); Steven K. LUNDY, Lake Forest Park, WA (US); Mark GILCHRIST, Lake Forest Park, WA (US); Jahan KHALILI, Kirkland, WA (US); Shi ZHUO, Chengdu, Sichuan (CN); Yong ZHANG, Chengdu, Sichuan (CN); Hai ZHU, Bothell, WA (US); Yi ZHU, Chengdu, Sichuan (CN)

(21) Appl. No.: 17/772,464

(22) PCT Filed: Feb. 10, 2021
(86) PCT No.: PCT/US2021/017305

§ 371 Date: Apr. 27, 2022

Related U.S. Application Data
(60) Provisional application No. 63/086,593, filed on Oct. 1, 2020.
(60) Provisional application No. 62/976,344, filed on Feb. 13, 2020.

(51) Int. Cl. C12N 9/48; A61P 31/14

(52) U.S. Cl. CPC C12N 9/485; C12Y 304/17023; A61P 31/14; C07K 2319/30

(57) ABSTRACT

A fusion protein, comprising a variant angiotensin converting enzyme 2 (ACE2) domain covalently fused to a Fc domain. The variant ACE2 domain has a N-terminal deletion, a C-terminal deletion, or both, relative to a full-length wildtype ACE2 having a SEQ ID NO: 1. The variant ACE2 domain has ACE2 activity.

### CROSS REFERENCE TO RELATED APPLICATIONS

This application claims the benefit of the filing date of U.S. Provisional Application Ser. No. 62/976,344 filed Feb. 13, 2020, and U.S. Provisional Application Ser. No. 63/086,593 filed Oct. 1, 2020 under 35 U.S.C. 119(e), the entire disclosures of which are incorporated by reference herein.

### TECHNICAL FIELD

The present application relates to the prevention or treatment of the diseases, symptoms or conditions involving Angiotensin-Converting Enzyme 2 (ACE2) such as coronavirus disease 2019 (COVID-19) and related conditions.

### BACKGROUND

Unless otherwise indicated herein, the materials described in this section are not prior art to the claims in this application and are not admitted to be prior art by inclusion in this section.

COVID-19 is an infectious disease caused by severe acute respiratory syndrome (SARS) coronavirus 2 (SARS-CoV-2). Complications of COVID-19 may include long-term lung damage, pneumonia, acute respiratory distress syndrome (ARDS), peripheral and olfactory nerve damage, multi-organ failure, septic shock, and death. A study of the first 41 cases of confirmed COVID-19, published in January 2020 in The Lancet, reported the earliest date of onset of symptoms as Dec. 1, 2019. By Mar. 11, 2020, the World Health Organization (WHO) declared the COVID-19 outbreak a pandemic. As of Sep. 26, 2020, more than 32.6 million cases have been reported across 188 countries and territories with more than 990,000 deaths, of which more than 7.5 million cases and 205,000 deaths were reported by the United States.

No medication or vaccine other than Remdesivir is approved with the specific indication to treat COVID-19. The US National Institute Health guidelines do not recommend any medication for prevention of COVID-19 outside the setting of a clinical trial, either before or after exposure to the SARS-CoV-2 virus. Nine vaccines have been authorized by at least one national regulatory authority for public use: two RNA vaccines from Pfizer-BioNTech and Moderna; three conventional inactivated vaccines from Sinopharm, Bharat Biotech, and Sinovac; three viral vector vaccines from Sputnik V, Oxford-AstraZeneca, and Janssen; and one peptide vaccine (EpiVacCorona).

Angiotensin-converting enzyme 2 (ACE2) is a zinc-containing metalloenzyme located on the cell membrane of mainly alveolar cells of the lung, enterocytes of the small intestine, endothelial cells of arterial and venous, smooth muscle cells of arteries, and other lineages of cells in the lungs, arteries, heart, kidney, intestines, and other tissues. ACE2 regulates the renin angiotensin system by counterbalancing angiotensin-converting enzyme activity in the cardiovascular, renal and respiratory systems, indicating its important role in the control of blood pressure. ACE2 plays a protective role in the physiology of hypertension, cardiac function, heart function, and diabetes. In the acute respiratory distress syndrome (ARDS), ACE, AngII, and AT1R promote the disease pathogenesis, whereas ACE2 and AT2R protect from ARDS. In addition, ACE2 has been identified as a receptor of severe acute respiratory syndrome (SARS) coronavirus and plays a key role in severe acute respiratory syndrome (SARS) pathogenesis. Of a family of coronaviruses, at least three viruses, SARS-CoV, MERS CoV, and SARS-CoV-2, use one of their viral proteins, also known as Spike, to bind to the ACE2 protein on the surface of human host cells for the viral entry into human body.

SARS-CoV-2 is one of seven known coronaviruses to infect humans, including SARS-CoV-1 and MERS CoV viruses that caused the outbreak of SARS in Asia in 2003 and in Middle East in 2012. The immune response to SARS-CoV-2 virus involves a combination of the cell-mediated immunity and antibody production. Although more than 100 million people have recovered from COVID-19, it remains unknown if the natural immunity to SARS-CoV-2 virus will be long-lasting in individuals. One of the concerns relates to the virus's continual accumulation of mutations, which may alter the spectrum of viral antigenicity and cause reinfection by mutant strains of the virus. As of January 2021, variant strains of SARS-CoV-2 virus identified in Europe and South Africa seem to be spreading so quickly. These variant strains may harbor mutations that ultimately enhance viral recognition and infection into host cells. Whether these or other foreseeable variants might diminish the potency of vaccines or overcome natural immunity and lead to a spate of reinfections remains unknown.

The other concern relates the phenomenon of antibody-dependent enhancement (ADE). ADE occurs when the binding of suboptimal antibodies enhances viral entry into host cells. In coronaviruses, antibodies targeting the viral spike (S) glycoprotein promote ADE. In cases of SARS-CoV-1 viruses, the antibodies that neutralized most variants were found to be able to enhance immune cell entry of the mutant virus, which, in turn, worsen the disease the vaccine was designed to protect against. Therefore, ADE can hamper vaccine development, as a vaccine may cause the production of suboptimal antibodies. In this context, any preventive strategy other than vaccines shall be considered as a viable alternative circumventing ADE, either before or after exposure to SARS-CoV-2 virus.

Therefore, there remains a significant need for effective treatments or preventions of the diseases or conditions involving Angiotensin-Converting Enzyme 2 (ACE2).

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides, among others, fusion proteins, fusion protein complexes, protein complexes, immunoconjugates containing fusion protein complexes and pharmaceutical compositions containing fusion protein complexes. The application also provides methods of making the fusion proteins and fusion protein complexed and methods for using fusion proteins or fusion protein complexes to treat or prevent diseases.

In one aspect, the application provides fusion proteins that have ACE2 activity. In one embodiment, the fusion protein includes a variant angiotensin converting enzyme 2 (ACE2) domain covalently fused to a Fc domain. In one embodiment, the variant ACE2 domain comprises a N-terminal deletion, a C-terminal deletion, or both, relative to a full-length wildtype ACE2. In one embodiment, the full-length wildtype ACE2 domain has an amino acid sequence with at least 95%, 97%, or 98% sequence identity to SEQ ID NO: 1. In one embodiment, the variant ACE2 domain has ACE2 activity.

In one embodiment, the variant ACE2 domain comprises an amino acid sequence having at least 75%, 80%, 85%, 90%, 95%, 97%, 98%, or 99% sequence identity to a segment of amino acid sequence from a full-length wildtype ACE2. In one embodiment, the segment may start with an amino acid residual selected from the residual 1-17 of a full-length wildtype ACE2. In one embodiment, the segment may end with an amino acid residual selected from the residual 615-740 of the full-length wildtype ACE2. For example, the variant ACE2 domain may have an amino acid sequence having at least 98% or 99% sequence identity to a segment of amino acid sequence from residual 1 to residual 615, from residual 2 to residual 618, from residual 2 to residual 740, from residual 4 to residual 615, from residual 17 to residual 615, from residual 17 to residual 740, or any other combination of the starting residual and ending residual, from a full-length wildtype ACE2.

In one embodiment, the variant ACE2 domain comprises an amino acid sequence having at least 75%, 80%, 85%, 90%, 95%, 97%, 98%, or 99% sequence identity to SEQ ID NO: 3.

In one embodiment, the variant ACE2 domain may have a higher binding affinity to SARS-CoV, or SARS Spike protein than the full-length wildtype ACE2. For example, the variant ACE2 domain may have a binding affinity to SARS-CoV, or SARS spike protein with a KD from 0.1 nM to 100 nM.

In one embodiment, the variant ACE2 domain may have a higher binding avidity to SARS-CoV, or SARS Spike protein than the full-length wildtype ACE2. For example, the variant ACE2 domain may have a binding avidity to SARS-CoV, or SARS spike protein with a KD from 0.01 nM to 10 nM.

In one embodiment, the Fc domain is derived from a Fc domain of an immunoglobulin. The immunoglobulin may be IgG1, IgG2, IgG3, IgG4, IgA1 (d-IgA1, 5-IgA1), IgA2, IgD, IgE, or IgM. In one embodiment, the Fc domain may have a Fc hinge region. In one embodiment, the Fc hinge region may be engineered to C220S. In one embodiment, the Fc domain may include a null mutation selected from K322A, L234A, and L235A when compared to a wildtype Fc domain. In one embodiment, the wildtype Fc domain has an amino acid sequence having at least 98%, or 99% sequence identity to SEQ ID NO: 5.

In one embodiment, the Fc domain may lack effector function. In one embodiment, the Fc domain may lack antibody-dependent cellular cytotoxicity (ADCC), antibody-dependent cellular phagocytosis (ADCP), and complement-dependent cytotoxicity (CDC). In one embodiment, the Fc domain comprises an IgG1 Fc domain.

In one embodiment, the Fc domain comprises an amino acid sequence having at least 75%, 80%, 85%, 90%, 95%, 97%, 98%, or 99% sequence identity to SEQ ID NO: 6.

In one embodiment, the fusion protein may have an amino acid sequence having at least 75%, 80%, 85%, 90%, 95%, 97%, 98%, or 99% of sequence identity to SEQ ID NOs: 7, 9, 11, 13, 15, 16, 17, 18, 19, and 21.

In one embodiment, the fusion protein may have a molecular weight from about 50 kDa to 250 kDa. In one embodiment, the fusion protein may have a molecule weight of 50 kDa, 60 kDa, 70 kDa, 80 kDa, 90 kDa, 100 kDa, 120 kDa, 150 kDa, 180 kDa, 200 kDa, 250 kDa or any number in between.

In a second aspect, the application provides fusion protein complexes. In one embodiment, the fusion protein complex is a homodimer of the fusion protein as disclosed herein. In one embodiment, the fusion protein complex includes two variant ACE2 domains.

In one embodiment, the fusion protein complex comprises at least two fusion proteins. In one embodiment, the two fusion protein are paired through one or two disulfide bonds. In one embodiment, the disulfide bond is located on the hinge of the Fc domain.

In one embodiment, the fusion protein or fusion protein complex has a binding affinity to SARS-CoV-2, SARS-CoV, or SARS spike protein or a fragment thereof. In one embodiment, the binding affinity has an equilibrium dissociation constant not greater than 0.1 nM, 0.5 nM, 1 nM, 2 nM, 3 nM, 5 nM, 10 nM, 20 nM, 25 nM, 30 nM, 40 nM, 50 nM, 60 nM, 80 nM, or any number in between.

In one embodiment, the fusion protein or fusion protein complex has a binding avidity to SARS-CoV-2, SARS-CoV, or SARS spike protein or a fragment thereof. In one embodiment, the binding avidity has an equilibrium dissociation constant not greater than 0.01 nM, 0.05 nM, 1 nM, 2 nM, 3 nM, 5 nM, 10 nM, or any number in between.

In one embodiment, the fusion protein or fusion protein complex has a specific enzymatic activity from about from 50 pmol/min/μg to about 5000 pmol/min/μg. In one embodiment, the fusion protein has a specific enzymatic activity of about 568 pmol/min/μg.

In a third aspect, the application provides protein complexes. In one embodiment, the protein complex includes the fusion protein or fusion protein complex as disclosed thereof bound to a viral protein. In one embodiment, the viral protein comprises SARS-CoV-2, SARS-CoV, SARS spike protein, coronavirus, SARS virus, or a fragment or a combination thereof.

In a further aspect, the application provides isolated nucleic acid encoding the fusion protein as disclosed thereof.

In a further aspect, the application provides an expression vector comprising the isolated nucleic acid encoding the fusion protein as disclosed thereof.

In a further aspect, the application provides host cell comprising the nucleic acid that encodes the fusion protein as disclosed thereof. In one embodiment, the host cell is a prokaryotic cell. In one embodiment, the host cell is an eukaryotic cell.

In a further aspect, the application provides methods for producing the fusion protein and fusion protein complex as disclosed thereof. In one embodiment, the method comprises culturing the host cell with nucleic acid encoding the fusion protein or fusion protein complex so that the fused protein or fusion complex is produced.

In a further aspect, the application provides protein-conjugate. In one embodiment, the protein-conjugate includes the fusion protein or fusion protein complex as disclosed thereof and a drug moiety. The drug moiety may be linked to the fusion protein or fusion protein complex through a linker. In one embodiment, the linker may be a covalent bond selected from an ester bond, an ether bond, an amine bond, an amide bond, a disulphide bond, an imide bond, a sulfone bond, a phosphate bond, a phosphorus ester bond, a peptide bond, a hydrazone bond or a combination thereof.

In one embodiment, the drug moiety may be an antiviral agent, an immune regulatory reagent, an imaging agent or a combination thereof. In one embodiment, the antiviral agent may be favipiravir, ribavirin, galidesivir, remdesvir, or a combination thereof. In one embodiment, the imaging agent may be radionuclide, a florescent agent, a quantum dots, or a combination thereof.

In a further aspect, the application provides pharmaceutical compositions for treating disease or condition involving Angiotensin-Converting Enzyme 2 (ACE2). In one embodiment, the pharmaceutical composition includes the fusion protein or fusion complex as disclosed herein and a pharmaceutically acceptable carrier. In one embodiment, the pharmaceutical composition further includes an antiviral agent. In one embodiment, the pharmaceutical composition includes the protein-conjugate as disclosed thereof and a pharmaceutically acceptable carrier.

In a further aspect, the application provides method of treating or preventing a viral infection, acute respiratory distress syndrome, pulmonary arterial hypertension, or acute lung injury in a subject. In one embodiment, the method includes the step of administering to the subject an effective amount of the fusion protein or fusion complex as disclosed herein. In one embodiment, the method further includes co-administering an effective amount of a therapeutic agent. In one embodiment, the therapeutic agent includes an antiviral agent. In one embodiment, the subject is a mammal.

In one embodiment, the viral infection may be the infection of SARS-CoV-2, SARS-CoV, SARS Spike protein, coronavirus, SARS virus, or a fragment or a combination thereof.

In one embodiment, the method may include administering the fusion protein or fusion protein complex intravenously, subcutaneously, through nasal passage (such as nasal spray), or through pulmonary passageway.

In a further aspect, the application provides a solution. In one embodiment, the solution includes an effective concentration of the fusion protein or fusion protein complex as disclosed herein. In one embodiment, the solution is blood plasma in a subject. In one embodiment, the solution includes the fusion protein, the fusion protein complex or the protein complex as disclosed herein, and the solution is blood plasma in a subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows (1A) the diagram of recombinant fusion proteins between ACE2 functional domain and engineered Fc (null) fragment (SI-69R2 and SI-69R4), (1B) the sequence of the SI-F019 fusion protein, a post-translational modified SI-69R2 devoid of N-terminal 17-amino acid signal peptide, (1C) the size-exclusion chromatograph indicating that the SI-F019 fusion protein complex is a homodimer, and (1D) the diagram of SI-F019-Spike protein complex;

FIG. 2 shows that SI-F019, but not SI-69R4, is resistant to TMPRSS2-dependent hydrolysis (2A), and that the enzymatic activity of SI-F019 can be quantified in an in vitro fluorometric assay (2B);

FIG. 3 demonstrates that SI-F019 dose-dependent blockade of live SARS-CoV-2 infection to VeroE6 cells has reached 100% at all three MOI of virus in the test;

FIG. 4 shows that the addition of SI-F019 at 10 fM or above protected a portion of Vero E6 cells from undergoing cell lysis after 1-hour of viral infection by either SARS-CoV-2 or SARS-CoV-1 viruses at a MOI of 0.01;

FIG. 5 shows that after preincubation with pseudo-virus, SI-F019 inhibits viral infection in a dose-dependent fashion and achieves a complete inhibition at higher concentrations (IC50=32.56 nM);

FIG. 6 shows the results of internalization/infection mediation assay that there was no uptake of GFP signals, indicative of pseudovirus (PsV), when pretreated with SI-F019 in the concentrations tested, while low GFP signals were associated with SI-69C1 (anti-S1 antibody) and SI-69R3 (SARS-CoV-2 ACE-2 Fc WT), as well as media, buffer, and ACE2-his (SI-69C1), at 48 hours in THP1 (pH 7.2)(6A), THP1 (pH 6.0)(6B), and Daudi (6C);

FIG. 7 shows that SI-F019 can compete against either a natural anti-SARS-CoV-2 antibody or an ACE2-Fc (wild type) fusion protein to block the Fc mediated antibody-dependent enhancement (ADE) as measured by GFP signals, indicative of PsV infection;

FIG. 8 shows the flow cytometry analysis of the HEK293-T cells expressing SARS-CoV-2 Spike protein as detected by using anti-Spike antibody and anti-human Fc antibody;

FIG. 9 shows the dose-dependent binding of SI-F019 to the HEK293-T cells expressing SARS-CoV-2 Spike protein as measured by geometric mean fluorescent intensity (gMFI);

FIG. 10 displays the FACS analysis of antibody-dependent cellular cytotoxicity (ADCC) assay showing that a human anti-S1 antibody (SI-69C3) directs human NK cells to target the HEK293-T cells expressing SARS-CoV-2 Spike protein, as measured by Calcein-AM and Propidium Iodide staining;

FIG. 11 shows that when compared to a human anti-S1 antibody (SI-69C3), SI-F019 did not mediate ADCC at the treatment doses between 100 fM and 100 nM, whereas its variant with wild type Fc (SI-69R3) did in a dose-dependent fashion, even though the level of activity was lower;

FIG. 12 shows that the Fc null mutations enable SI-F019 to reduce the serum-mediated complement-dependent cytotoxicity (CDC) in vitro as measured by the viability of HEK293-T cells expressing SARS-CoV-2 S protein;

FIG. 13 shows that SI-F019 does not induce serum complement-dependent cytotoxicity (CDC) in vitro by measuring the viability of HEK293-T cells expressing SARS-CoV-2 S protein after various treatments (13A); and the Fc null mutations of SI-F019 have no effect on the subsequent cell growth at 96 hours post treatment in vitro; and

FIG. 14 shows that SI-F019 does not elicit the release of cytokines in PBMC culture in either soluble or plate-bound form: (14A) IFNγ; (14B) TNFα; (14C) GM-CSF; (14D) IL-2; (14E) IL-10; (14F) IL-6; (14G) IL-1β; (14H) IL-12p70; and (141) MCP-1.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present application relates to, among others, the generation and characterization of fusion proteins such as recombinant human ACE2-Fc fusion proteins. In some embodiments, these fusion proteins are capable of protecting the membranous ACE2 of human host cells from the viral particles or virus. In one embodiment, the viral particles or virus may utilize viral spike proteins for viral entry into host cells after infection. In one embodiment, the viral particles include, but not limited to, SARS-CoV-2 virus, COVID-19 virus, variants of SARS-CoV-2, and other coronaviruses. In one embodiment, the virus may cause severe acute respiratory syndrome (SARS). In one embodiment, the SARS may include coronavirus disease 2019 or COVID-19.

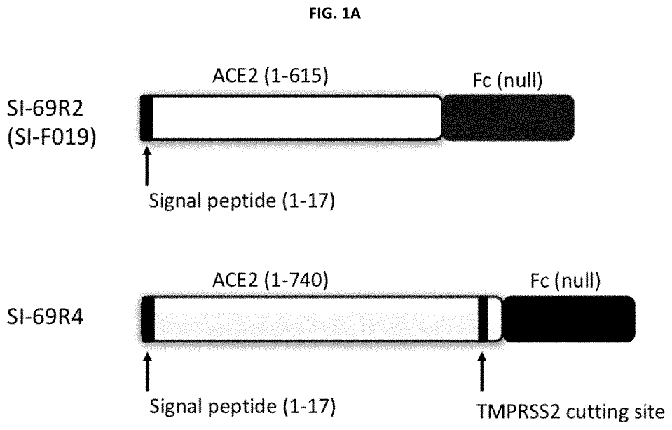

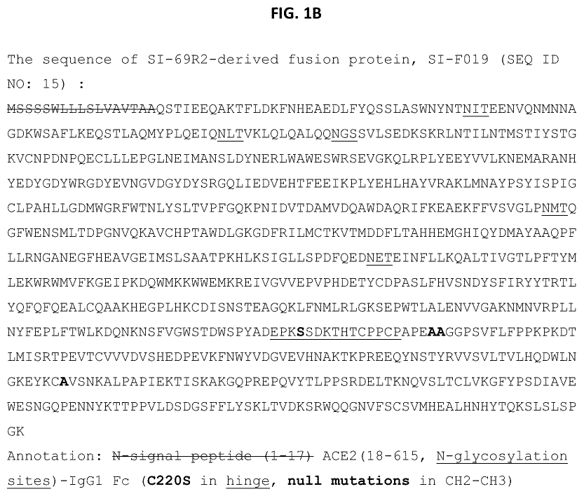

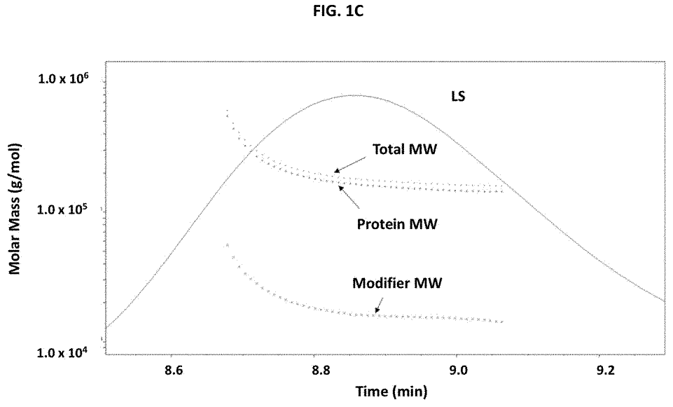

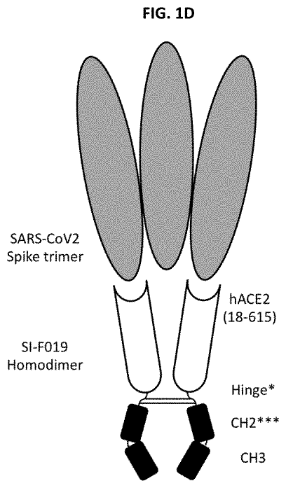

In one embodiment, the recombinant human ACE2-Fc fusion proteins may be a fusion protein of ACE2 zinc metallopeptidase domain (also known as ACE2 extracellular domain, ACE2-ECD) and IgG1 Fc fragment. In one embodiment, the fusion protein is SI-F019, a fusion protein of ACE2-ECD and IgG1 Fc fragment with mutations of C220S, L234A, L235A, and K322A according to EU numbering system (Table 1 and FIG. 1). An active ACE2-ECD retains the structural conformation for the host receptor-virus interaction. Each mutation in IgG1 Fc fragment may deplete certain immune responses. The mutation C220S may remove unpaired cysteine for pairing heavy and light chains and thus providing the technical advantage of, among others, avoiding protein forming aggregate, improving protein stability and promoting manufacture efficiency and scalability. The introduction of both L234A and L235A may reduce the effector function of Fc, such as antibody-dependent cellular cytotoxicity (ADCC), antibody-dependent cellular phagocytosis (ADCP). The K322A mutation may reduce C1q binding triggered complement-dependent cytotoxicity (CDC). SI-F019 is designed to neutralize SARS-CoV-2 virus while trigging fewer effector response.

The terms “a”, “an” and “the” as used herein are defined to mean “one or more” and include the plural unless the context is inappropriate.

The term “recombinant fusion protein” refers to a protein that is created through genetic engineering of a fusion gene encoding two or more genes that originally coded for separate proteins.

The term “ACE2-Fc” refers to a recombinant fusion protein of a human ACE2 protein fragment and an engineered fragment of the fragment crystallizable region (Fc region) of a human immunoglobulin, where the human Immunoglobulin including, but not limited to, IgG1, IgG2, IgG3, IgG4, IgA1 (d-IgA1, S-IgA1), IgA2, IgD, IgE, and IgM.

The term “spike”, “Spikes”, “S protein”, or variants refers to the protein responsible for allowing the virus to attach (“S1 subunit” or “S1 protein”) to and fuse (“S2 subunit” or “S2 protein”) with the membrane of a host cell. In the case of COVID-19, SARS-CoV-2 has sufficient affinity to the ACE2 receptor on human cells to use them as a mechanism of cell entry, and SARS-CoV-2 has a higher affinity to human ACE2 than the original SARS virus.

The term “Fc domain”, “Fc fragment”, and “Fc region” refer to the identical domain or fragment of the Fc region (“Fc domain” and “Fc fragment”, respectively) in IgG, IgA, and IgD antibody isotypes, which is derived from the hinge, and the second and third constant domains (CH2-CH3) of the antibody's two heavy chains.

The term “affinity” refers to a measure of the attraction between two polypeptides, such as receptor/ligand, ACE2/spike protein or it's variants, for example. The intrinsic attractiveness between two polypeptides can be expressed as the binding affinity equilibrium constant (KD) of a particular interaction. A KD binding affinity constant can be measured, e.g., by Bio-Layer Interferometry.

The term “avidity” refers to the accumulated strength of multiple affinities of individual non-covalent binding interactions, such as between a protein receptor and its ligand, and is commonly referred to as functional affinity. As such, avidity is distinct from affinity, which describes the strength of a single interaction.

The term “antigenic drift” refers to random genetic mutation of an infectious virus resulting in a new strain of virus with minor changes in antigenicity, to which the antibodies that prevented infection by previous strains may not be effective.

The term “cytokine release syndrome” (CRS) refers to CRS in severe cases of COVID-19 associated with an increased level of inflammatory mediators including cytokines and chemokines, such as interleukin (IL)-2, IL-6, IL-7, IL-10, tumor necrosis factor (TNF), granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, ferritin, and D-dimers in blood upon SARS-CoV-2 infection.

### EXAMPLES

### Example 1. The Cloning, Expression, and Purification of Recombinant ACE2-Fc Fusion Proteins

Human membranous ACE-2 is the receptor critical for mediating SARS-CoV viral entry into host cells in human. The human ACE2 protein has at least three functional domains: a signal peptide (residues 1-17), zinc metallopeptidase domain (residues 18-615), and a TMPRSS2 protease cutting site (residues 697-716) (SEQ ID NO: 1 is the full length human ACE2 protein sequence from Genbank number: NP_001358344.1), of which the SARS-CoV viral protein, Spike, interacts with the zinc metallopeptidase domain (SEQ ID NO: 3 is the protein sequence of truncated ACE2 from residue 1 to 615). On the other hand, the Fc region of a human antibody (SEQ ID NO: 5) is capable of interacting with Fc receptors (FcRs) on many immune cells and some proteins of the complement system. Each Fc fragment of IgG1 Fc region contains a cysteine at C220 (according to EU numbering system), which may intrinsically form disulfide bond with either kappa or lambda light chain. To reduce the risk of having a free cysteine that may destabilize and/or inactivate the protein, C220 may be substituted for serine (C220S) or other amino acids. To reduce the Fc binding to FcγR and C1q, other point mutations, such as K322A, L234A, and L235A, may be engineered into wild type IgG1 Fc fragment. Collectively, the IgG1 Fc fragment harboring the four mutations is called IgG1 Fc null (SEQ ID NO: 6).

The recombinant human ACE2-Fc fusion proteins (as listed in Table 1) were engineered to produce soluble fusion proteins, of which SI-69R2 (SEQ ID NO: 7) is a recombinant fusion protein of a truncated ACE2 fragment without the TMPRSS2 protease cutting site and the IgG1 Fc null fragment. Other recombinant fusion proteins were created to provide a Fc fragment of Ig isotype, such as SI-69R2-G4 (IgG4 Fc, SEQ ID NO: 9), SI-69R2-A1 (IgA1 Fc, SEQ ID NO: 11), SI-69R2-A2(IgA2 Fc, SEQ ID NO: 13), or wild type IgG1 Fc fragment (IgG1 Fc, SEQ ID NO: 19). The recombinant fusion protein of a truncated ACE2 with all three domains and a wild type IgG1 Fc fragment was also created (SI-69R4, 1-740, SEQ ID NO: 21). Of all recombinant ACE2-Fc fusion proteins, the signal peptide (ACE2 residues 1-17) may be replaced with other signal peptides at different lengths, without affecting the function of other domains in either human ACE protein or ACE2-Fc fusion proteins.

The recombinant fusion genes encoding the fusion proteins in Table 1 were cloned into either pCGS3.0 (such as SI-69R2) or pTT5 expression vector (such as SI-69R4 and SI-69R10) and expressed in ExpiCHO cells. All the fusion proteins were purified following standard protein expression protocols, sterilized using a 0.22 um filter, and stored in a cryopreservation buffer at 4° C. During the expression and purification, each recombinant fusion protein may undergo post-translational modification, including N-glycosylation and the cleavage of N-terminal signal peptide (17 amino acids). In case of SI-69R2, the purified fusion protein was given a new name, SI-F019.

As shown in FIGS. 1A and 1B, SI-F019 retains the truncated ACE2 fragment (residues 18-615) encompassing the zinc metallopeptidase domain (residues 19-611) of human ACE2 but not the TMPRSS2 protease cutting site. In addition, SI-F019 retains the IgG1 Fc null fragment devoid of its binding to Fcγ receptors. In this way, SI-F019 in its soluble form is not expected to bind any target cells in peripheral blood.

The SI-F019 fusion protein likely undergoes post-translation modification, such as N-glycosylation, and homodimerization linked by the two disulfide bonds of Fc region. To assess the actual molecule weight of the SI-F019 dimer, the analytical size exclusion chromatography (SEC) was used, in a combination of multi-angle light scattering (MALS), absorbance (UV), and/or refractive index (RI) concentration detectors techniques, as shown in FIG. 1C. The method combines the chromatographic separation by molecular size and the determination of absolute molar mass by light scattering (LS) without the limitations of molecule weight standard calibration. SI-F019 exhibited an average total molecular weight of 209.6 kDa (main peak), of which the molecule weights of the SI-F019 dimer and its modifiers (i.e. glycans) were measured at 189.3 kDa and 20.3 kDa, respectively. In the theoretical calculation of its amino acids, the molecule weight of the SI-F019 monomer is 95.1 kDa. Thus, the purified SI-F019 fusion protein complex is a homodimer, whereas SI-F019 protein complex refers to the protein-protein interaction between SI-F019, as either a monomer or a dimer, and other proteins, such as spike proteins and effector proteins. The formation of SI-F019-Spike protein complex (as illustrated in FIG. 1D) underlies the mechanism by which SI-F019 is a candidate inhibitor for preventing SARS-CoV-2 virus from docking onto the membranous ACE2 for viral entry into the host cells in human.

### Example 2: The Binding Affinity of SI-F019 to Spikes, Fc Receptors, and C1q

SI-F019 was designed to block SARS-CoV viral entry into human by preventing the spike proteins from binding to the membranous ACE2 protein on human host cells. Spikes are the most distinguishing feature of coronaviruses, which are the knoblike structures responsible for the corona- or halo-like surface. The spike proteins are generally composed of glycoproteins, and each spike is composed of a trimer of the S protein, and the S protein is in turn composed of an S1 and S2 subunit. The homotrimeric S protein mediates the receptor binding and membrane fusion between the virus and host cell. The S1 subunit forms the head of the spike and has the receptor-binding domain (RBD). The S2 subunit forms the stem which anchors the spike in the viral envelope and on protease activation enables fusion. In a functionally active state, the subunit complex of S1 and S2 is split into individual subunits when the virus binds and fuses with the host cell under the action of proteases, such as cathepsin family and transmembrance protease serine 2 (TMPRSS2) of the host cell. Spikes play important roles in the viral entry of infection process by coronavirus. In case of COVID-19, SARS-CoV-2 virus docks onto the membrane bound ACE2 receptor on the host cell surface, and the interaction between spikes and the functional domain of ACE2 brings about the release of viral nucleocapsid into the host cell cytoplasm by triggering fusion between the viral envelope and host cell membranes.

SI-F019 was evaluated for the binding affinity and avidity of ACE-Fc fusion proteins to the viral spike proteins. In a Bio-Layer Interferometry analysis, the samples of spike proteins include SARS-CoV-2 spike trimer, SARS-CoV-2 S1 protein, SARS-CoV-2 S1 protein RBD domain, and SARS-CoV-1 RBD domain (Table 2). The binding affinity assay measured the binding of SI-F019 immobilized on the anti-human IgG Fc Capture Biosensors tip surface to the spike protein in solution. The avidity assay measured the binding of a biotinylated spike protein immobilized on the Streptavidin Biosensors tip surface to SI-F019 in solution. The data analysis utilized a 1:1 fitting model to calculate both the binding affinity and avidity. The result indicates that the binding affinity and avidity of SI-F019 to these spike proteins, fragments, or domains seem to be within their respective scales of KD in nanomolar (nM) (Table 2). This characteristic and informative data may be useful references for measuring the SI-F019 protein complex with variants of viral spike proteins indicative of potential antigenic drift among SARS-CoV-2 variants. The phenomenon is close to be reality referring to the viral mutations in certain strains of SARS-CoV-2 virus, such as D614G in the spike protein (Zhang et al., 2020), which has likely altered the viral affinity to membranous ACE2 and viral entry into the host cells.

In parallel to its binding to spikes, SI-F019 was evaluated for its binding to human FcγRs, C1q, and FcRn by using Bio-Layer Interferometry. As shown in Table 3, the binding to FcγRs, including FcγRI, FcγRIIa, FcγRIIb, and FcγRIIIa, was not detected, nor the binding to C1q. However, SI-F019 did bind to FcRn and the binding affinity was determined at a KD of 37.6 nM, which is comparable to that of human IgG1 Fc region.

### Example 3. SI-F019 is Resistant to TMPRSS2 Protease Activity

Human ACE2 is subject to membranous protease hydrolysis by TMPRSS2, and monomeric extracellular ACE2 is shed from cells, which can be readily detected in serum. In the recombinant ACE2-Fc fusion proteins, the truncated ACE2 domain is fused to Fc fragment but still retains the binding affinity to the viral spike proteins.

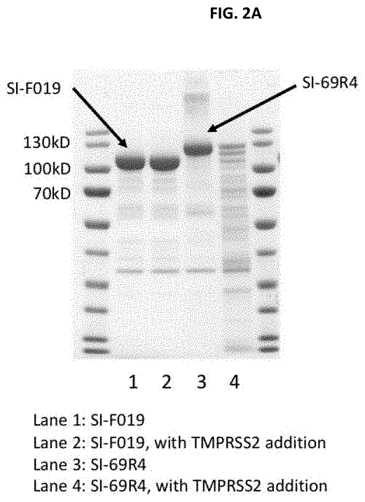

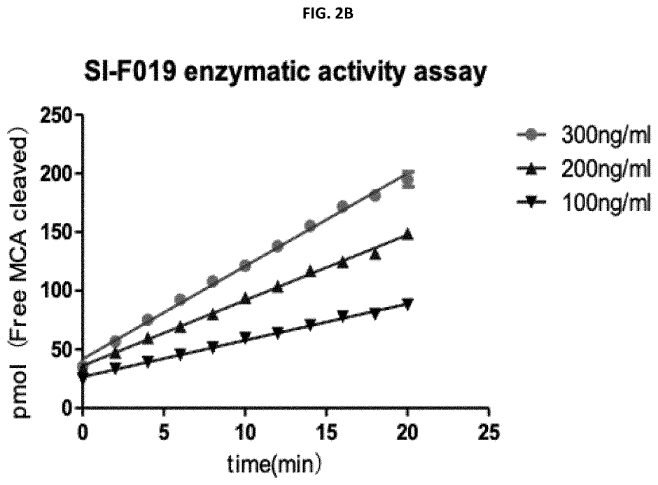

SI-F019 was engineered without the TMPRSS2 cutting site in the truncated ACE2 domain. As shown in FIG. 1, SI-F019 contains residues from 18 to 615, whereas SI-69R4 encodes all three ACE2 domains (residue: 1-740, SEQ ID NO: 21) encompassing the TMPRSS2 cutting site. To demonstrate that SI-F019 is free from TMPRSS2-specific proteolysis, SI-69R4 was used as a control. To carry out the assay of TMPRSS2-specific hydrolysis, the TMPRSS2 (106-492) catalytic domain was cloned, expressed, and purified according to Genbank: NP_001358344.1. As shown in FIG. 2A, in the absence of TMPRSS2, both SI-F019 and SI-69R4 stably migrated to their respective molecule weights (as monomers under denature condition). When TMPRSS2 was added, SI-F019 revealed its resistance to TMPRSS2, whereas SI-69R4 underwent proteolysis indicating its sensitivity to TMPRSS2 as predicted. Thus, SI-F019 is stable and is resistant to TMPRSS3-mediated protease activity.

### Example 4. SI-F019 Exerts the Enzymatic Activity of ACE2

SI-F019 is a fusion protein of a truncated ACE2 (residue 18-615) and IgG1 Fc null fragment. The truncated ACE2 encodes a zinc metallopeptidase, whose enzymatic activity may be re-evaluated by using an established assay. A peptide substrate of ACE2 with an MCA (7-Methoxycoumarin-4-acetic acid) fluorescent tag [MCA-YVADAPK (Dnp)-OH Fluorogenic Peptide Substrate] was used to measure ACE2 enzymatic activity of SI-F019. MCA molecule was prepared as standard curve calibration for free fluorophore quantification, and the substrate was diluted in DMSO to 0.97 mg/ml. SI-F019 was diluted to 100, 200, and 300 ng/ml and used to cleave fluorogenic peptide in-vitro to release free MCA. The assay was incubated at room temperature for 20 minutes, and data were collected for fluorescent signals at timepoints with 2 minute intervals.

The cleaved MCA was quantified in molar using MCA standard curve. The enzymatic activity was determined according to the slope of linear curve as shown in FIG. 2B (MCA quantity against time). SI-F019 showed good linearity (R²>0.99) at all three concentrations, indicating that the stable cleavage of peptide was concentration-dependent. For calculating the enzymatic activity, the slope was divided by mass number (μg) of SI-F019. The final specific enzymatic activity was 568 pmol/min/μg. The fact that SI-F019 retains the enzymatic activity of the membranous ACE2 indicates that this independent domain of ACE2 also retains the structural conformation for the host receptor-virus interaction.

### Example 5. SI-F019 Inhibits Live SARS-CoV-2 Infection to VeroE6 Cells

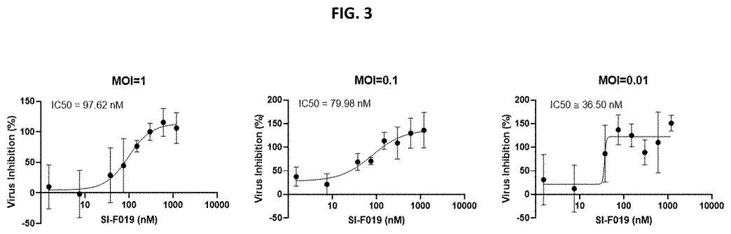

SI-F019 was tested for the ability to inhibit live SARS-CoV-2 infection and lysis of VeroE6 (ATCC: CRL-1586) cells in vitro. SI-F019 test concentrations, ranging from 1.5 nM to 1200 nM, were preincubated with 3 concentrations of live SARS-CoV-2 virus (Strain USA-WA1/2020, representing a 100-fold range of Multiplicity Of Infection, MOI) for 1 hour and then added to 90% confluent monolayer of VeroE6 cells. After 1 hour, the medium containing the virus was removed and replaced with the medium containing SI-F019 at matching test concentrations, and the tests were conducted in triplicate. The cell viability was measured by neutral red dye uptake after 72 hours and the percentage of inhibition of lytic viral infection was determined by comparison to wells in which virus was added at each MOI without SI-F019. The 50% inhibitory concentrations (IC50) for each virus concentration (1 MOI=40,000 virus particles) were calculated using GraphPad Prism software and are shown on each graph. The preincubation of SI-F019 with live SARS-CoV-2 resulted in a dose-dependent blockade of infection that reached 100% at all three MOI of virus that were tested. As shown in FIG. 3, SI-F019 neutralized as much as 40,000 virus particles at an MOI of 1.0, with an IC50 of 97.62 nM. At MOI of 0.1 and 0.01, SI-F019 was able to block the infection at IC50 of 79.95 nM and 36.5 nM, respectively.

### Example 6. SI-F019 Reduces Virus Replication and Reinfection

SI-F019 was tested for its ability to inhibit replication and reinfection, i.e. further transfer of infection to VeroE6 cells from the cells previously infected with a low MOI of SARS-CoV-2 or SARS-CoV-1 viruses. VeroE6 cells in a 90% confluent monolayer (^{˜}20,000 cells) were exposed to either SARS-CoV-2 (Strain USA-WA1/2020) or SARS-CoV-1 (Strain Urbani 2003000592) for 1 hour at an MOI of 0.01 (calculated as 400 virus infective particles). After washing out free virus particles, SI-F019 was added to the cells in a range from 10 fM to 100 nM in triplicates and the cell culture was maintained for 72 hours. Cell viability was determined by neutral red dye uptake and % inhibition of viral cytotoxicity was calculated. Absorbance values were normalized on each plate using the maximum absorbance of the conditions with no virus or no drug (NVND) representing 100% cell viability, and the average absorbance value of the virus/no drug (VND) establishing the maximum cell death using the formula:

% Cell Survival=[(Well OD₅₄₀−VND OD₅₄₀)/(NVND OD₅₄₀−VND OD₅₄₀)]*100

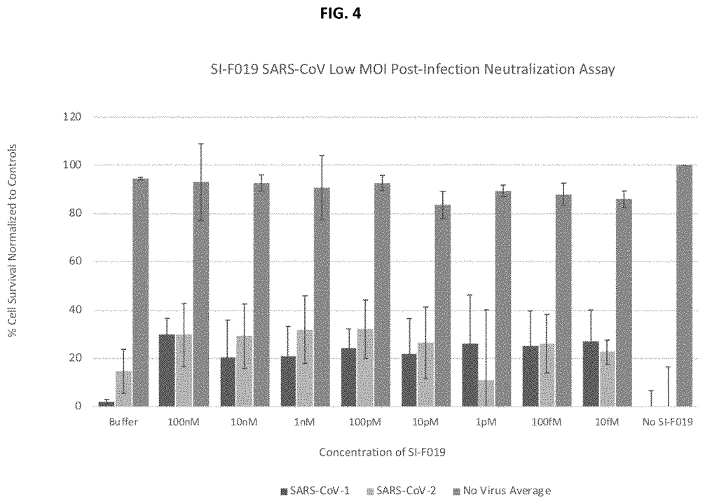

As shown in FIG. 4, the addition of SI-F019, at a concentration of 10 fM protected Vero E6 cells from secondary infection. The culture infected with either SARS-CoV-2 or SARS-CoV-1 virus at an MOI of 0.01 for 1-hour reduced the cell lysis by at least 20%. However, no significant increase in protection was observed in this assay when the concentration of SI-F019 was increased by 10-fold increments up to 100 nM. The finding indicates that, to the cells infected with a low titer of virus, the addition of SI-F019 may reduce the spread of virus as well as the degree of cytotoxicity, even at low concentrations.

### Example 7. SI-F019 Inhibits Pseudo-Virus Infection of HEK293T-ACE2 Cells

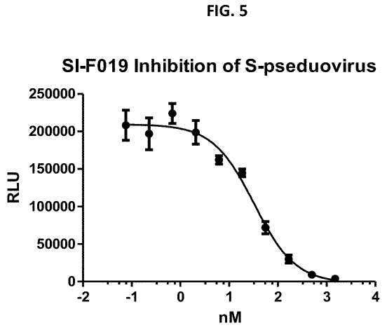

HEK293T (ATCC: CRL-3216)-3D4 clone cell line was generated by lentiviral transduction of human ACE2 protein. The function of expressed human ACE2 was confirmed by enzymatic substrate conversion assay and binding by specific antibody by FACS. SARS-CoV-2 S protein packaged pseudo-virus which containing a luciferase reporter gene was obtained from National Institute for the Control of Pharmaceutical & Biological Products. Testing was conducted according to the manufacturer's instructions. The S-pseudo virus stock solution was diluted in culture medium with MRD of 20 in order to yield 300 TCID50/well of virus load. SI-F019 at concentrations ranging from 0.07 nM to 1500 nM were preincubated with the diluted virus solution for 1 hour. HEK293T-3D4 cells were dispersed into a 96-well plate. After 1 hour, mixtures were added into cell plate. Infected cells were measured by testing luciferase activity after 24 hours of incubation. 50% inhibitory concentrations (IC50) for defined virus load were calculated using GraphPad Prism software. FIG. 5 shows that after preincubation with pseudo-virus, SI-F019 inhibits viral infection in a dose-dependent fashion and achieves a complete inhibition at higher concentrations (IC50=32.56 nM).

### Example 8. SI-F019 Reduces the Incidence of ADE

Antibody-dependent enhancement (ADE) is a phenomenon in which binding of a virus to suboptimal antibodies enhances its entry into host cells. In case of COVID-19, the secondary infection of SARS-CoV-2 virus to the patient who has anti-SARS-CoV-2 antibodies developed from a primary infection or to an individual who has been vaccinated may lead to enhanced uptake of virus by monocytes and B cells. The anti-virus antibodies in contact with the virus may bind to Fc receptors expressed on certain immune cells or some of the complement proteins. The latter binding depends on the Fc region of the antibody. Typically, the virus undergoes degradation in a process called phagocytosis, by which viral particles are engulfed by host cells through plasma membrane. However, the antibody binding might result in virus escape if the virus is not neutralized by an antibody, either due to low affinity binding or targeting a non-neutralizing epitope. Then, the outcome is an antibody enhanced infection.

The antibodies developed through either natural immunity or vaccination possess a wild type Fc region. While SI-F019 is capable of competing with anti-spike antibodies for binding to SARS-CoV2 virus, the IgG1 Fc null fragment is incapable of binding to either Fc receptors or C1q (see Table 3). To demonstrate its comparative advantage in reducing the effect of ADE, SI-F019 was evaluated for its role in internalization, replication, and reinfection.

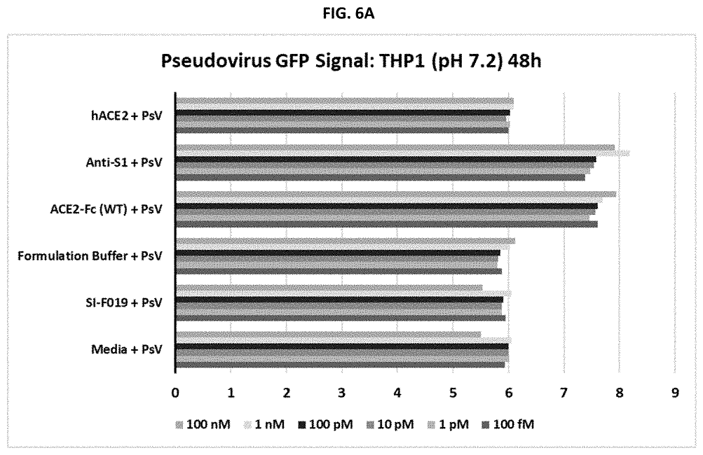

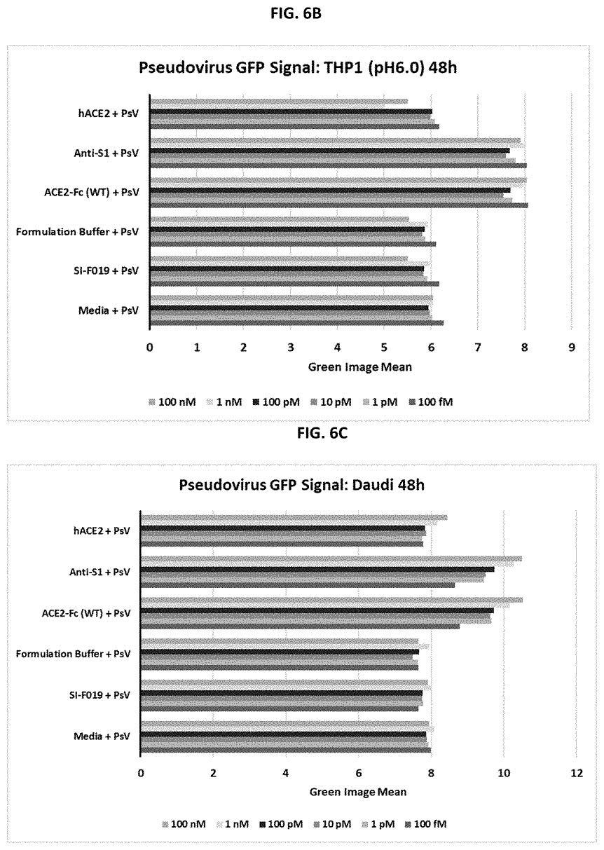

In an assay for measuring Fc mediated internalization, the SARS-CoV-2 S protein was packaged into GFP-expressing pseudo-virus (PsV), and two cell lines, THP1 (monocyte) and Daudi (B cell) that express Fc receptors and complement receptor 2 (CR2), were used for testing FcRγ and CR2-mediated ADE mechanisms. SI-69R3 was used as a control for SI-F019, having a wild type Fc in contrast to SI-F019 that has an IgG1 Fc null modification (see Table 1). After being exposed to PsV for 48 hours, the green fluorescent signal from the cells was quantified as an indicator of PsV infection. In the conditions treated with PsV and SI-69C1, anti-S1 antibody, or SI-69R3 low levels of green fluorescence were measured at 48h in THP1 (pH 7.2) (6A), THP1 (pH 6.0)(6B), and Daudi (6C) cells. This result indicated that some transfer of PsV could occur via the Fc receptor. In contrast, the condition with SI-F019 at the indicated concentrations resulted in no uptake of PsV by THP1 or Daudi cells, comparable with the green fluorescent signal measured in the negative control conditions including, assay media, formulation buffer, and SI-69C1(FIG. 6). The effect of Fc mediated ADE was dose-dependent, as the treatment to the cells was carried out using doses ranging from 1 pM up to 100 nM. This indicates that some uptake of PsV may occur through either FcγR or CR2 mechanisms.

### Example 9. SI-F019 Reduces Virus Load of PsV

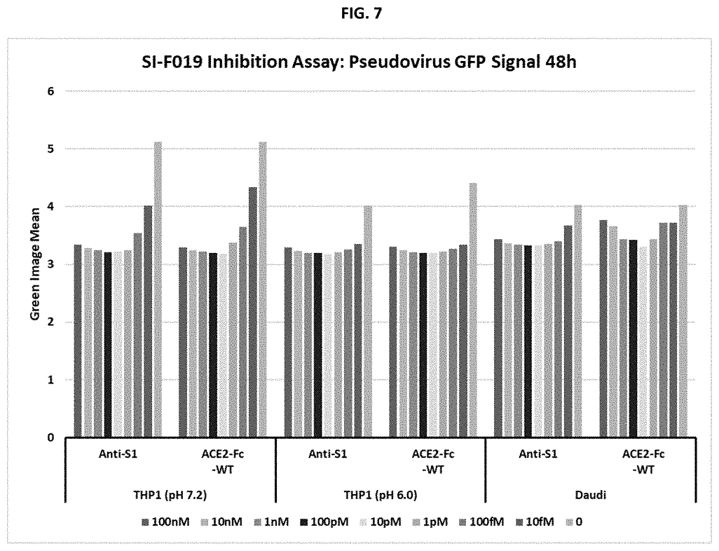

SI-F019 may not mediate the internalization of S protein packaged GFP-expressing pseudo-virus (PsV) due to lack of a functional Fc fragment. To determine if SI-F019 can inhibit the uptake of the pseudovirus, SI-F019 was used as a co-treatment with either SI-69R3 or natural anti-SARS-CoV-2 antibody in a competition mode. The PsV was incubated for 1 hour with SI-F019 at a dose range from 1 pM to 100 nM, together with either 10 pM of anti-SARS-CoV-2 (51) antibody or 10 pM of SI-69R3 prior to infecting the same set of target cells. PsV derived GFP signals were detected as the virus load of infection. SI-F019 was able to inhibit the virus load of PsV in the target cells starting at 10 fM (FIG. 7).

While both the antibody, such as anti-SARS-CoV-2 (51) antibody, and the fusion protein of truncated ACE2-wild type Fc fragment in SI-69R3, were shown to be able to mediate internalization of SARS-CoV-2 Spike pseudotyped lentivirus, SI-F019 failed to do so due to lack of a functional Fc fragment. Herein, SI-F019 helped reduce virus load of PsV in the presence of either 10 pM of anti-SARS-CoV-2 (51) antibody or 10 pM of SI-69R3, even at a low concentration of 10 fM. Together, these results indicate that SI-F019 may reduce the incidence of ADE induced by FcRγ and CR2 dependent mechanisms in THP1 monocytes and Daudi B cells, respectively.

### Example 10. HEK293-T Cells Expressing SARS-CoV-2 Spike Protein

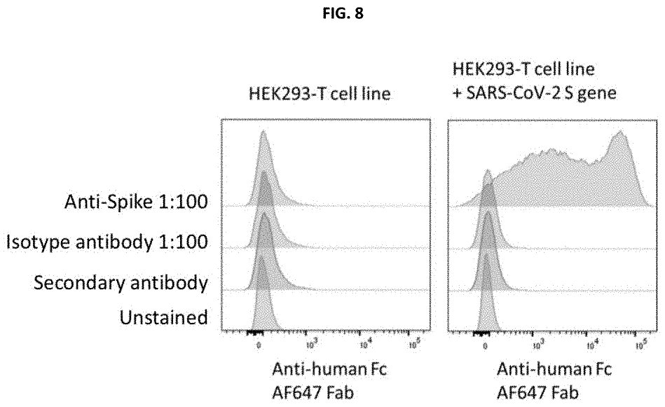

HEK293-T cells (ATCC: CRL-3216) that stably express SARS-CoV-2 spike protein were established by transducing the lentivirus packaged with SARS-CoV-2 spike protein encoding cDNA (Accession: YP_009724390.1) and IRES expression and selection based on puromycin resistance driven by same expression construct (LPP-CoV219-Lv105-050, GeneCopoeia). The expression of SARS-CoV-2 spike protein was confirmed by binding of a human IgG clone AM001414, specific for SARS-CoV-2 Spike protein “Anti-Spike”, (SKU938701, Biolegend) and the Human IgG Isotype matched clone QA16A12 was used as control “Isotype”, (SKU403502, Biolegend). Bound protein was quantified by secondary incubation with polyclonal anti-human Fc AF647 Fab (SKU109-607-008, Jackson ImmunoResearch) and FACS evaluation as shown in FIG. 8.

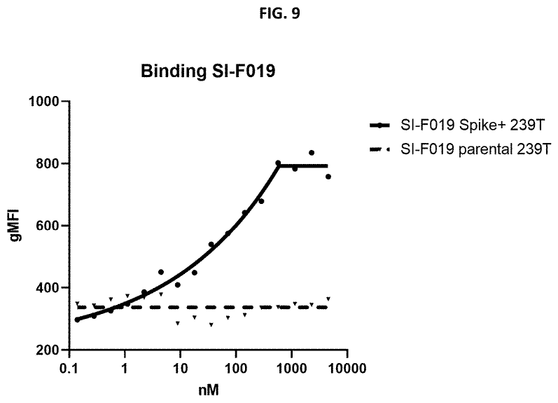

HEK293-T cells expressing either SARS-CoV-2 spike protein and the parental HEK293 cells were stained with the indicated materials for 30 minutes at 37° C. in the presence of internalization inhibitor sodium azide. After the removal of free SI-F019, SI-F109 was detected and quantified by using anti-human Fc AF647 fab (SKU109-607-008, Jackson ImmunoResearch) and flow cytometry analysis. Geometric mean signal intensity was used to quantify the binding of SI-F019 and target cells line as shown in FIG. 9. HEK293-T cells expressing either SARS-CoV-2 spike protein may serve as a model for COVID-19 infected cells.

### Example 11. The Effect of SI-F019 on Antibody-Dependent Cellular Cytotoxicity (ADCC)

Antibody-dependent cellular cytotoxicity (ADCC) is one of important immune responses to viral infection, such as the infection of SARS-CoV-2 virus in the case of COVID-19. Following the initial viral infection, anti-virus antibodies directly bind to the viral particles for neutralization and agglutination. Binding of a virus-antibody complex to an Fc receptor on a phagocyte can trigger phagocytosis, resulting in destruction of the virus; binding to the Fc receptors on immune effector cells, such as monocytes, neutrophils, eosinophils and NK cells, can trigger the release of cytotoxic factors (e.g., antiviral interferons), creating a microenvironment that is hostile to virus replication.

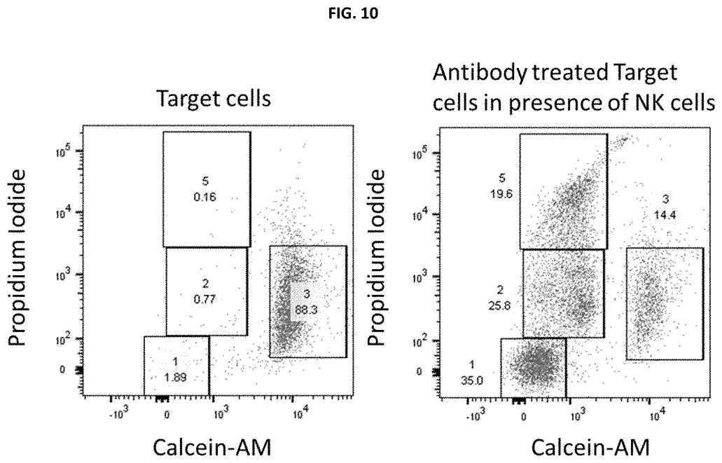

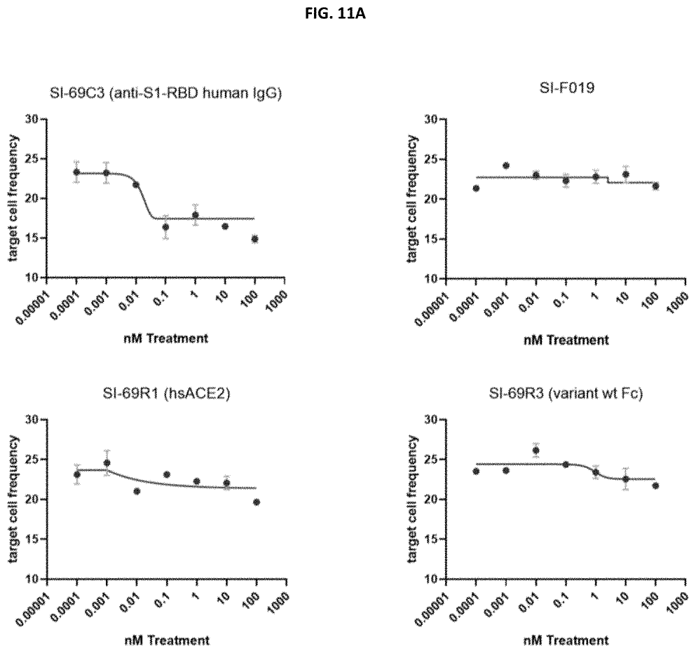

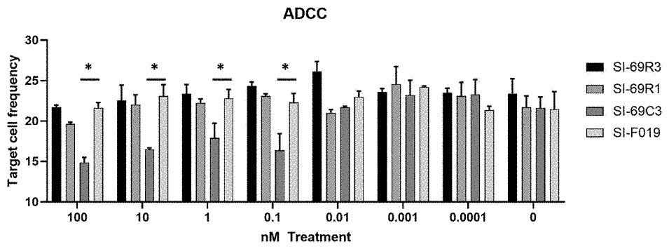

To distinguish the effect of SI-F019 from anti-spike antibodies, HEK293-T cells expressing SARS-CoV-2 spike protein were loaded with Calcein-AM and co-cultured with purified human NK cells at a 5:1 effector to target ratio. Treatments tested included SI-F019 and S1-specific human IgG clone SI-69C3. SI-69C3 is the human antibody clone CC12.3, isolated from a hospitalized COVID-19 patient (10.1126/science.abc7520). After 12 hours in co-culture, cells were stained with propidium iodide and evaluated for viability. As shown in FIG. 10, reduction in the viable target cell frequency (Population 3) based on expression of Calcein-AM and Propidium Iodide staining was evaluated as a measure of cytolysis.

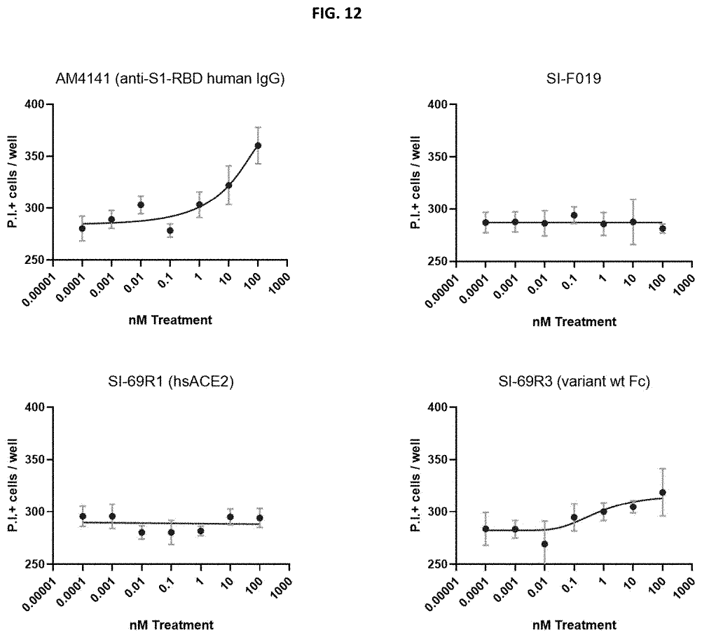

ADCC mediated by NK cells can be directed toward HEK293-T cells expressing SARS-CoV-2 protein when exposed to SI-specific human IgG clone SI-69C3 (Clone CC12.3). SI-F019 did not mediate ADCC compared to SI-69C3 within the treatment range of 100 nM to 100 fM. Under these assay conditions, the SI-F019 drug variant with wt Fc (SI-69R3) was able to mediate ADCC in a dose-dependent fashion, but the level of activity was lower compared to the S1-specific human IgG clone CC12.3 as shown in FIG. 11 and FIG. 12. These data indicate that unlike SARS-CoV-2 S1 specific human IgG antibodies, SI-F019 does not mediate NK cell-mediated ADCC.

### Example 12. The Effect of SI-F019 on Complement-Dependent Cytotoxicity (CDC)

The role of the complement cascade in mediation of antibody-based cell and tissue injury in COVID-19 patients is evident in both the natural immune responses and neutralizing antibody-based therapy (Perico et al., 2021). Immune complexes formed of virus and specific IgG mediate complement-induced blood clotting, thromboembolism and systemic microangiopathy. These widespread complications in COVID-19 patients can be life-threatening and are dependent on the complement proteins binding to IgG. Virus immune complexes bridging red blood cells through C1q and platelets with FcγRIIA are mediators of the thromboembolism in COVID-19 patients (Nazy et al., 2020). The fixation of immune complexes to endothelial vessel walls and complement-mediated coagulation are a primary concern in patients with COVID-19 where the activation of endothelial cells is part of the thromboembolism cascade.

Unlike a natural IgG antibody, SI-F019 is unable to binding C1q as shown in Table 3. This feature eliminates the risk of the induction of cell death of infected epithelia and endothelium that may transiently express the SARS-CoV-2 spike protein on their surface. This protective effect of SI-F019 is demonstrated in comparison to anti-spike human IgG antibody.

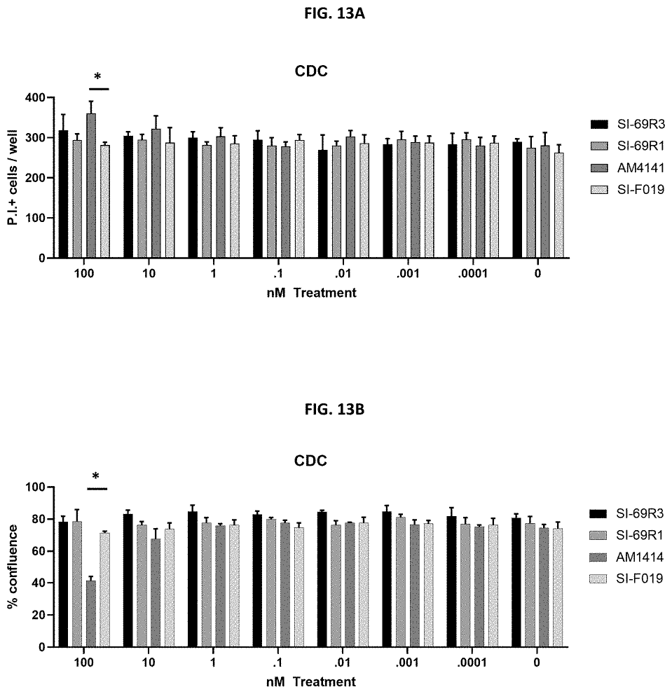

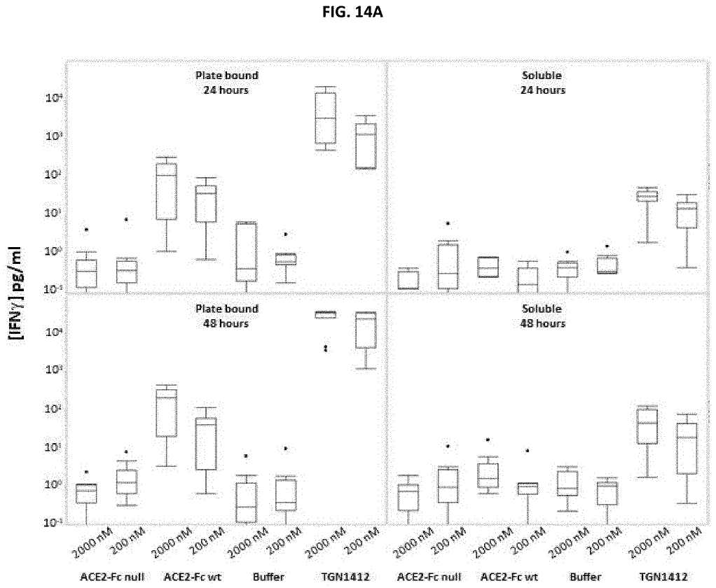

To demonstrate the protective effect of SI-F019, HEK293-T cells expressing SARS-CoV-2 Spike protein were cultured in serum-free media (Optimem) with treatments for 30 minutes, followed by addition of human serum complement at 1:10 serum-to-media ratio. Treatments tested included SI-F019 and S1-specific human IgG clone AM001414. Cell were cultured at 37° C. for 3 hours prior to addition of Propidium Iodide staining and positive staining cells counted in each well. Red cells counted by Incucyte Zoom Software at 3 hours are evaluated as a measure of CDC as shown in FIG. 12 and FIG. 13. Total cellular confluence was evaluated after 96 hours as a measure of the impact of CDC as displayed in FIG. 13.

The protection of tissue cells from complement damage is further confirmed by the ability of these cells to further proliferate after human serum complement challenge. CDC mediated by human serum complement at a 1:10 volume to volume ratio with serum free media is evaluable toward HEK293T cells expressing SARS-Cov-2 S protein when exposed to S1-specific human IgG clone (Clone AM4141). The result indicated that both human soluble monomeric ACE2 and SI-F019 did not mediate CDC, whereas SI-69R3 had limited, dose dependent increase CDC activity compared to human IgG antibody. CDC cytolysis was reflected in reduced cell growth, based on well confluence at 96 hours post treatment.

### Example 13. Cytokine Release Elicited by Soluble or Plate-Bound SI-F019 in PBMC Culture

SARS-CoV-2 has a tropism for ACE2-expressing epithelia of respiratory tract and small intestine. Clinical laboratory findings of elevated IL-2, IL-6, IL-7, granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1α), and tumor necrosis factor-α (TNF-α) indicative of cytokine release syndrome (CRS) suggest an underlying immunopathology. CRS is a major adverse side effect that can limit the utility of treatment with biologics and is tested for using in vitro cytokine release assays.

SI-F019 is a fusion protein consisting of human ACE2 and a mutated form of human IgG1 Fc that is incapable of binding to Fcγ receptors. As such, SI-F019 is not expected to bind any target cells in peripheral blood or to elicit cytokine release. White blood cells (WBC) including neutrophils, isolated from 5 healthy donors were put in culture wells containing either plate-bound or soluble SI-F019 at 2000 nM and 200 nM concentrations.

As a positive control, the TGN1412 antibody was used at the same concentrations and in the same formats due to its well-documented ability to induce cytokine release in the plate-bound format of this assay. The potential contribution of the IgG1 Fc null fragment to reduce cytokine release was evaluated by comparison with SI-69R3 having a wild type Fc fragment that is capable of binding Fcγ receptors expressed by several cell types in peripheral blood. WBC cultures containing only the formulation buffer for SI-F019 at similar dilutions were used as a negative control. Culture supernatants were collected at 24 and 48-hour time points and the presence of 9 cytokines was detected using the Meso Scale Discovery (MSD) platform.

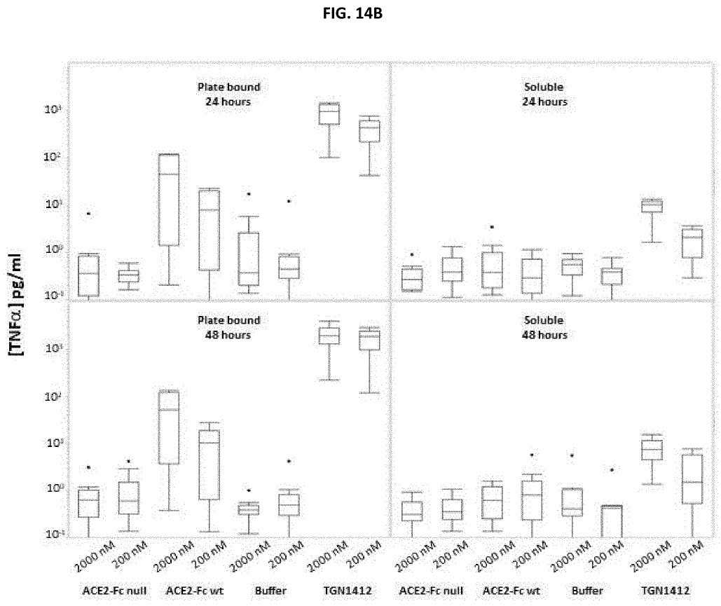

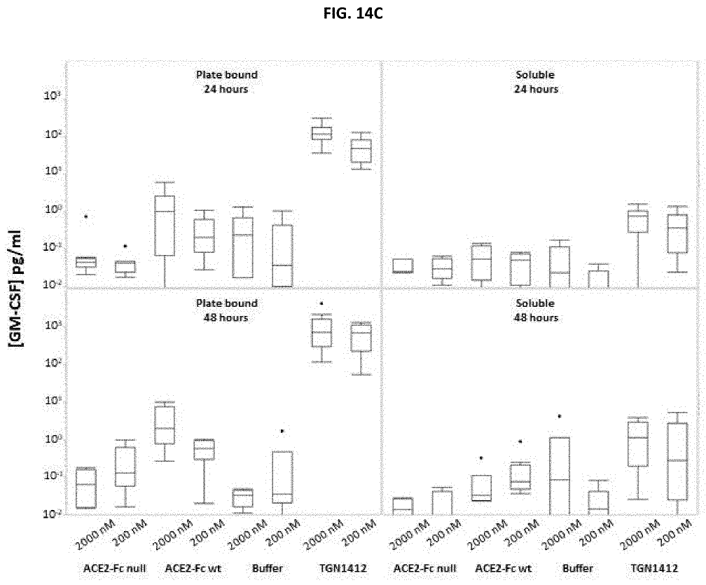

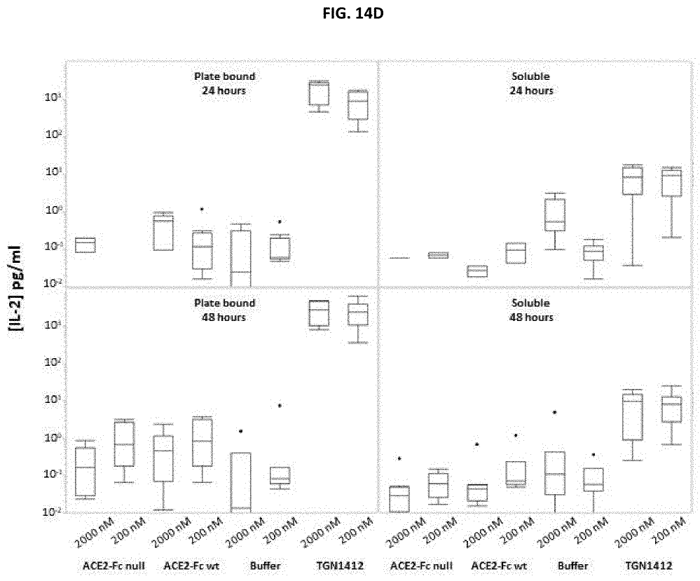

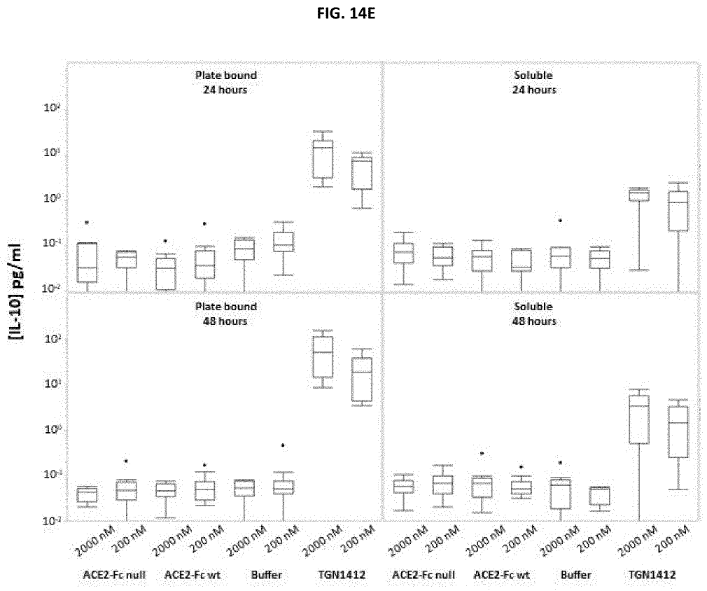

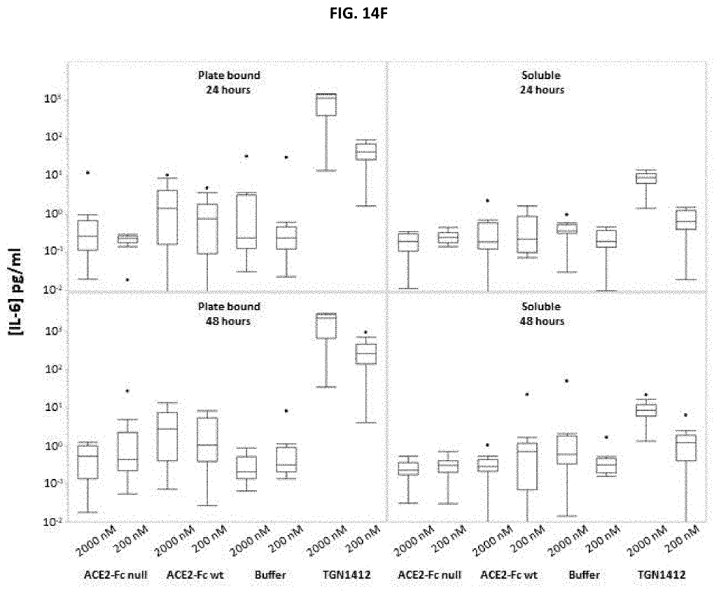

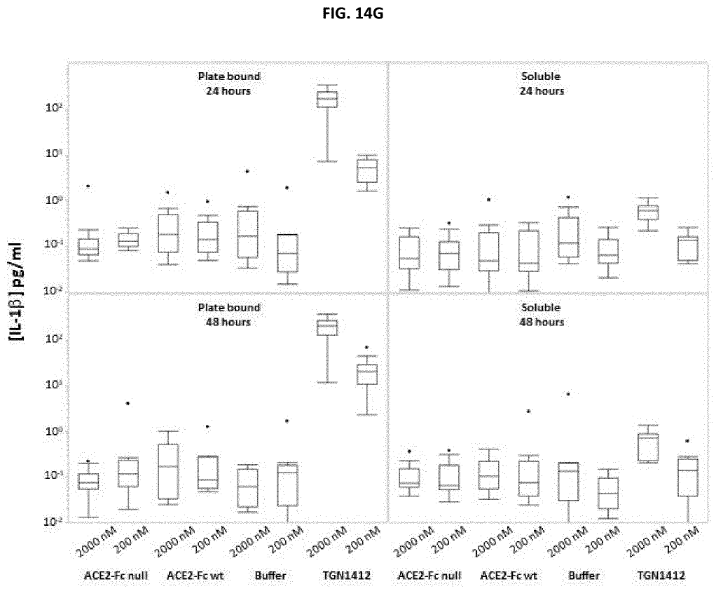

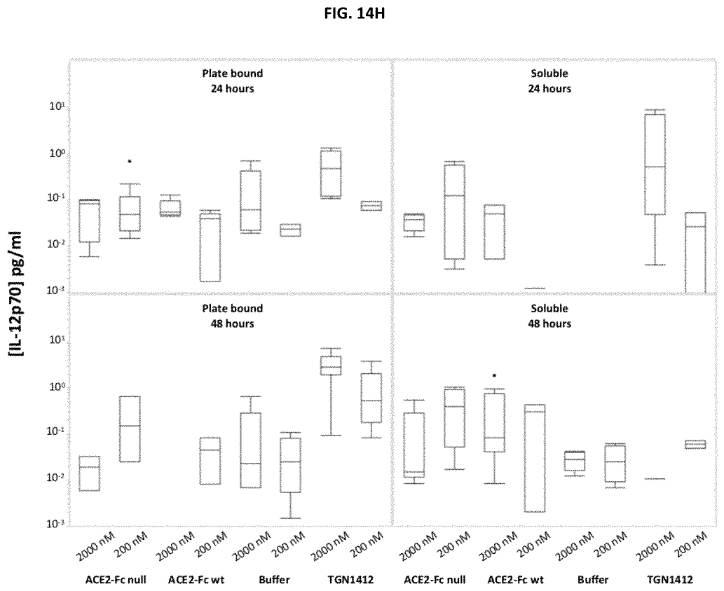

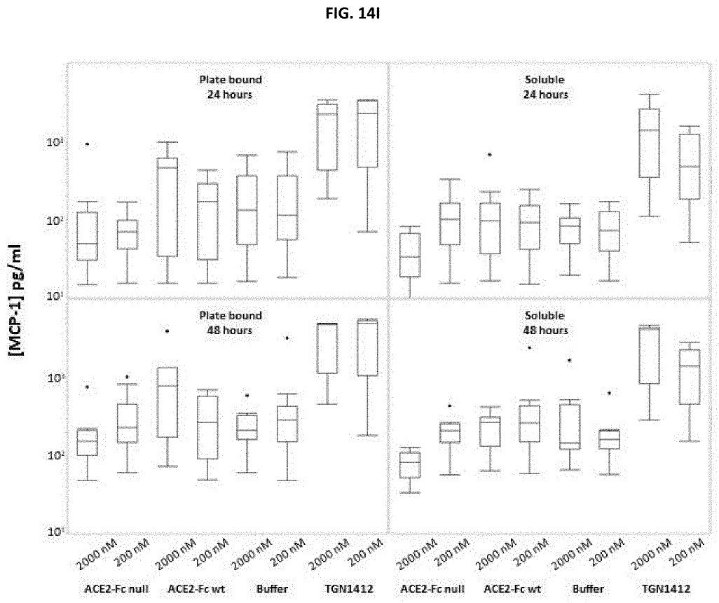

Included in the cytokine panel were the T cell-associated cytokines IFNγ, TNFα, GM-CSF, IL-2 and IL-10 as shown in FIGS. 14A-14E. Also tested were levels of the proinflammatory, non-T cell associated cytokines IL-1β, IL-12p70 and IL-6, as well as the monocyte chemoattractant protein, MCP-1 as shown in FIGS. 14F-141. Results from duplicate wells for each blood donor were averaged and plotted using JMP14 software in box plots showing the 95% confidence intervals and outliers.

The results indicate that SI-F019 does not induce any of the tested cytokines from exposed to WBC in either plate-bound or soluble formats at 200 nM and 2000 nM concentrations. Cytokine levels in SI-F019 treated samples showed concentrations similar to buffer controls in all conditions. The positive control, TGN1412 strongly induced most of the cytokines in the plate-bound but not the soluble format, which is in an agreement with previously published results. Some intermediate production of IFNγ, GM-CSF, and TNFα were detectable when plate-bound ACE2-Fc wild type was used to stimulate the WBC indicating the increased safety of the Fc null fragment of SI-F019.

The pathogenic role of the humoral response against SARS-CoV-2 virus has recently been suggested in patients receiving interventional IgG therapy (Weinreich et al., 2021; Chen et al., 2021). The small vessel hyperinflammatory response underlies adverse events, including thrombocytosis, pruitus, pyrexia, and hypertension. The present application demonstrates that SI-F019 could provide the benefit of virus neutralization comparable to that of IgG therapy while protecting tissues and organs from multiple pathways of dysfunctions. Therefore, SI-F019 may be used for treating, preventing, or moderating a viral infection, specifically for preventing and managing the progression of COVID-19 with reduced clinical complications, and additionally for acute respiratory distress syndrome, pulmonary arterial hypertension, or acute lung injury.

### Tables

TABLE 1
The cloning, expression, and purification of recombinant ACE2-Fc fusion proteins

| Recombinant fusion protein | Sample ID | | Purified fusion protein |
|---|---|---|---|
| huACE2 (1-615) - 6His-tagged | SI-69R1 | | huACE2 (18-615) - 6His-tagged |
| huACE2 (1-615) - IgG1 Fc (w2) | SI-69R3 | | huACE2 (18-615) - IgG1 Fc (w2) |
| huACE2 (1-740) - IgG1 Fc (w2) | SI-69R4 | | huACE2 (18-740) - IgG1 Fc (w2) |
| huACE2 (1-615) - IgG1 Fc null | SI-69R2 | SI-F019 | huACE2 (18-615) - IgG1 Fc null |

| | | |
|---|---|---|
| huACE2 (1-615) - IgG4 Fc | SI-69R2-G4 | huACE2 (18-615) - IgG4 Fc |
| huACE2 (1-615) - IgA1 Fc | SI-69R2-A1 | huACE2 (18-615) - IgA1 Fc |
| huACE2 (1-615) - IgA2 Fc | SI-69R2-A2 | huACE2 (18-615) - IgA2 Fc |

TABLE 2
The affinity and avidity of SI-F019 binding to viral proteins

| | Affinity | Kon | Kdis | Avidity | Kon | Kdis |
|---|---|---|---|---|---|---|
| | (nM) | (1/ms) | (1/s) | (nM) | (1/ms) | (1/s) |
| CoV2 S1 | 14.7 | 3.37E+05 | 4.93E−03 | 0.29 | 1.49E+05 | 4.72E−05 |
| CoV2 RBD | 13.8 | 3.45E+05 | 4.79E−03 | 0.89 | 3.09E+05 | 2.70E−04 |
| CoV RBD | 14.0 | 4.26E+05 | 5.97E−03 | 0.33 | 5.18E+05 | 1.71E−04 |
| COV2 SPIKE | NA | NA | NA | 0.18 | 4.20E+04 | 1.12E−05 |
| Trimer | | | | | | |

TABLE 3
The effect of Fc null mutations on its binding to Fc receptors

| Fc Receptor | KD (nM) | Kon (1/ms) | Kdis (1/s) |
|---|---|---|---|
| FcγRI/CD64 | Not detectable | | |
| FcγRIIa/CD32a | Not detectable | | |
| FcγRIIb/CD32b | Not detectable | | |
| FcγRIIIa/CD16a | Not detectable | | |
| C1q | Not detectable | | |
| FcRn | 37.6 | 4.51E+05 | 3.52E−0.2 |

### Sequence Listing

| | | SEQ ID NO: | |
|---|---|---|---|
| Sample ID | Annotation | Protein | DNA |
| huACE2 | huACE2 full length (Genbank_number: | 1 | 2 |
| wild type | Protein: NP_001358344.1; DNA: NM_021804.3) | | |
| huACE2 | huACE2 functional domain (residue: 1-615) | 3 | 4 |
| (1-615) | | | |
| | IgG1 Fc wild type | 5 | — |
| | IgG1 Fc null | 6 | — |
| SI-69R2 | A fusion protein of huACE2 (1-615) and IgG1 Fc null | 7 | 8 |
| SI-69R2-G4 | A fusion protein of huACE2 (1-615) and IgG4 Fc | 9 | 10 |
| SI-69R2-A1 | A fusion protein of huACE2 (1-615) and IgA1 Fc | 11 | 12 |
| SI-69R2-A2 | A fusion protein of huACE2 (1-615) and IgA2 Fc | 13 | 14 |
| SI-F019 | A fusion protein of huACE2 (18-615) and IgG1 Fc null | 15 | |
| | (purified from SI-69R2) | | |
| | A fusion protein of huACE2 (18-615) and IgG4 Fc | 16 | |
| | (purified from SI-69R2-G4) | | |
| | A fusion protein of huACE2 (18-615) and IgA1 Fc | 17 | |
| | (purified from SI-69R2-A1) | | |
| | A fusion protein of huACE2 (18-615) and IgA2 Fc | 18 | |
| | (purified from SI-69R2-A2) | | |
| SI-69R3 | A fusion protein of huACE2 (1-615) and IgG1 Fc (w2) | 19 | 20 |
| SI-69R4 | A fusion protein of huACE2 (1-740) and IgG1 Fc (w2) | 21 | 22 |
| SI-69R1 | 6His-tagged huACE2 (1-615) | 23 | 24 |
| SI-69R10 | 6His-tagged human TMPRSS2 protein (residue: 106-492) | 25 | 26 |
| | IgJ chain | 27 | — |
| | Secretory Component | 28 | — |

>Sequence ID 1: huACE2 full length protein sequence
(Genbank number: NP_001358344.1, TMPRSS2 protease cutting site)
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADQSIKVRISLKSALGDKAYEWNDNEM
YLFRSSVAYAMRQYFLKVKNQMILFGEEDVRVANLKPRISFNFFVTAPKNVSDIIPRTEVEKAI
RMSRSRINDAFRLNDNSLEFLGIQPTLGPPNQPPVSIWLIVFGVVMGVIVVGIVILIFTGIRDR
KKKNKARSGENPYASIDISKGENNPGFQNTDDVQTSF
>Sequence ID 2: huACE2 full length DNA sequence
(Genbank number: NM_021804.3)
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACCAAAGCATCAA
AGTGAGGATAAGCCTAAAATCAGCTCTTGGAGATAAAGCATATGAATGGAACGACAATGAAATG
TACCTGTTCCGATCATCTGTTGCATATGCTATGAGGCAGTACTTTTTAAAAGTAAAAAATCAGA
TGATTCTTTTTGGGGAGGAGGATGTGCGAGTGGCTAATTTGAAACCAAGAATCTCCTTTAATTT
CTTTGTCACTGCACCTAAAAATGTGTCTGATATCATTCCTAGAACTGAAGTTGAAAAGGCCATC
AGGATGTCCCGGAGCCGTATCAATGATGCTTTCCGTCTGAATGACAACAGCCTAGAGTTTCTGG
GGATACAGCCAACACTTGGACCTCCTAACCAGCCCCCTGTTTCCATATGGCTGATTGTTTTTGG
AGTTGTGATGGGAGTGATAGTGGTTGGCATTGTCATCCTGATCTTCACTGGGATCAGAGATCGG
AAGAAGAAAAATAAAGCAAGAAGTGGAGAAAATCCTTATGCCTCCATCGATATTAGCAAAGGAG
AAAATAATCCAGGATTCCAAAACACTGATGATGTTCAGACCTCCTTTTAG
>Sequence ID 3: huACE2 functional domain (residue: 1-615) protein
sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYAD
>Sequence ID 4: huACE2 functional domain (residue: 1-615) DNA
sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGAC
>Sequence ID 5: Fc wild type IgG1 Fc (EU numbering 216-447)
EPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
>Sequence ID 6: Fc null version (EU numbering 216-447, with
mutations: C220S, L234A, L235A, and K322A)
EPKSSDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQP
REPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
>Sequence ID 7: SI-69R2_huACE2 functional domain (residue: 1-
615) - IgG1 Fc (null) protein sequence (EU numbering 216-447,
with mutations: C220S, L234A, L235A, and K322A)
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADEPKSSDKTHTCPPCPAPEAAGGPSV
FLFPPKPKDTLMISRTPEVTCVVVDVSHEDPEVKFNWYVDGVEVHNAKTKPREEQYNSTYRVVS
VLTVLHQDWLNGKEYKCAVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEAL
HNHYTQKSLSLSPGK
>Sequence ID 8: SI-69R2: huACE2 functional domain (residue: 1-
615) - IgG1 Fc (null) DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACGAGCCCAAATC
TTCCGACAAAACTCACACATGCCCACCGTGCCCAGCACCTGAAGCCGCGGGGGGACCGTCAGTC
TTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCGCGGTCTCCAACA
AAGCCCTCCCAGCCCCCATCGAGAAAACCATCTCCAAAGCCAAAGGGCAGCCCCGAGAACCACA
GGTGTACACCCTGCCCCCATCCCGGGATGAGCTGACCAAGAACCAGGTCAGCCTGACCTGCCTG
GTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCAC
CGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATAG
>Sequence ID 9: huACE2 functional domain (residue: 1-615)- IgG4
Fc protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADESKYGPPCPPCPAPEFLGGPSVFLF
PPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNVFSCSVMHEALHNH
YTQKSLSLSLGK
>Sequence ID 10: huACE2 functional domain (residue: 1-615)- IgG4
Fc DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACGAGTCCAAATA
TGGTCCCCCGTGCCCACCATGCCCAGCACCTGAGTTCCTGGGGGGACCATCAGTCTTCCTGTTC
CCCCCAAAACCCAAGGACACTCTCATGATCTCCCGGACCCCTGAGGTCACGTGCGTGGTGGTGG
ACGTGAGCCAGGAAGACCCCGAGGTCCAGTTCAACTGGTACGTGGATGGCGTGGAGGTGCATAA
TGCCAAGACAAAGCCGCGGGAGGAGCAGTTCAACAGCACGTACCGTGTGGTCAGCGTCCTCACC
GTCCTCCACCAGGACTGGCTGAACGGCAAGGAGTACAAGTGCAAGGTCTCCAACAAAGGCCTCC
CGTCCTCCATCGAGAAAACCATCTCCAAAGCCAAAGGGCAGCCCCGAGAGCCACAGGTGTACAC
CCTGCCCCCATCCCAGGAGGAGATGACCAAGAACCAGGTCAGCCTGACCTGCCTGGTCAAAGGC
TTCTACCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAGGCTAACCGTGGACAA
GAGCAGGTGGCAGGAGGGGAATGTCTTCTCATGCTCCGTGATGCATGAGGCTCTGCACAACCAC
TACACGCAGAAGAGCCTCTCCCTGTCTCTGGGTAAATGA
>Sequence ID 11: huACE2 functional domain (residue: 1-615) - IgA1
Fc Protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDOWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADSQDVTVPCPVPSTPPTPSPSTPPTP
SPSCCHPRLSLHRPALEDLLLGSEANLTCTLTGLRDASGVTFTWTPSSGKSAVQGPPERDLCGC
YSVSSVLPGCAEPWNHGKTFTCTAAYPESKTPLTATLSKSGNTFRPEVHLLPPPSEELALNELV
TLTCLARGFSPKDVLVRWLQGSQELPREKYLTWASRQEPSQGTTTFAVTSILRVAAEDWKKGDT
FSCMVGHEALPLAFTQKTIDRLAGKPTHVNVSVVMAEVDGTCY
>Sequence ID 12: huACE2 functional domain (residue: 1-615) - IgA1
Fc DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACAGCCAGGATGT
GACTGTGCCCTGCCCAGTTCCCTCAACTCCACCTACCCCATCTCCCTCAACTCCACCTACCCCA
TCTCCCTCATGCTGCCACCCCCGACTGTCACTGCACCGACCGGCCCTCGAGGACCTGCTCTTAG
GTTCAGAAGCGAACCTCACGTGCACACTGACCGGCCTGAGAGATGCCTCAGGTGTCACCTTCAC
CTGGACGCCCTCAAGTGGGAAGAGCGCTGTTCAAGGACCACCTGAGCGTGACCTCTGTGGCTGC
TACAGCGTGTCCAGTGTCCTGCCGGGCTGTGCCGAGCCATGGAACCATGGGAAGACCTTCACTT
GCACTGCTGCCTACCCCGAGTCCAAGACCCCGCTAACCGCCACCCTCTCAAAATCCGGAAACAC
ATTCCGGCCCGAGGTCCACCTGCTGCCGCCGCCGTCGGAGGAGCTGGCCCTGAACGAGCTGGTG
ACGCTGACGTGCCTGGCACGCGGCTTCAGCCCCAAGGACGTGCTGGTTCGCTGGCTGCAGGGGT
CACAGGAGCTGCCCCGCGAGAAGTACCTGACTTGGGCATCCCGGCAGGAGCCCAGCCAGGGCAC
CACCACCTTCGCTGTGACCAGCATACTGCGCGTGGCAGCCGAGGACTGGAAGAAGGGGGACACC
TTCTCCTGCATGGTGGGCCACGAGGCCCTGCCGCTGGCCTTCACACAGAAGACCATCGACCGCT
TGGCGGGTAAACCCACCCATGTCAATGTGTCTGTTGTCATGGCGGAGGTGGACGGCACCTGCTA
CTGA
>Sequence ID 13: huACE2 functional domain (residue: 1-615) - IgA2
Fc Protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKOLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADSQDVTVPCRVPPPPPCCHPRLSLHR
PALEDLLLGSEANLTCTLTGLRDASGATFTWTPSSGKSAVQGPPERDLCGCYSVSSVLPGCAQP
WNHGETFTCTAAHPELKTPLTANITKSGNTFRPEVHLLPPPSEELALNELVTLTCLARGFSPKD
VLVRWLOGSQELPREKYLTWASRQEPSQGTTTYAVTSILRVAAEDWKKGETFSCMVGHEALPLA
FTQKTIDRMAGKPTHINVSVVMAEADGTCY
>Sequence ID 14: huACE2 functional domain (residue: 1-615) - IgA2
Fc DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACAGCCAGGATGT
GACTGTGCCCTGCCGAGTTCCCCCACCTCCCCCATGCTGCCACCCCCGACTGTCGCTGCACCGA
CCGGCCCTCGAGGACCTGCTCTTAGGTTCAGAAGCGAACCTCACGTGCACACTGACCGGCCTGA
GAGATGCCTCTGGTGCCACCTTCACCTGGACGCCCTCAAGTGGGAAGAGCGCTGTTCAAGGACC
ACCTGAGCGTGACCTCTGTGGCTGCTACAGCGTGTCCAGTGTCCTGCCTGGCTGTGCCCAGCCA
TGGAACCATGGGGAGACCTTCACCTGCACTGCTGCCCACCCCGAGTTGAAGACCCCACTAACCG
CCAACATCACAAAATCCGGAAACACATTCCGGCCCGAGGTCCACCTGCTGCCGCCGCCGTCGGA
GGAGCTGGCCCTGAACGAGCTGGTGACGCTGACGTGCCTGGCACGTGGCTTCAGCCCCAAGGAT
GTGCTGGTTCGCTGGCTGCAGGGGTCACAGGAGCTGCCCCGCGAGAAGTACCTGACTTGGGCAT
CCCGGCAGGAGCCCAGCCAGGGCACCACCACCTATGCTGTGACCAGCATACTGCGCGTGGCAGC
CGAGGACTGGAAGAAGGGGGAAACCTTCTCCTGCATGGTGGGCCACGAGGCCCTGCCGCTGGCC
TTCACACAGAAGACCATCGACCGCATGGCGGGTAAACCCACCCATATCAATGTGTCTGTTGTCA
TGGCGGAGGCGGACGGCACCTGCTACTGA
>Sequence ID 15: SI-F019_huACE2 functional domain (residue: 18-
615) - IgG1 Fc (null) protein sequence (with mutations at C220S,
L234A, L235A, and K322A, EU numbering)
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDOWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADEPKSSDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKC
AVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLSLSPGK
>Sequence ID 16: huACE2 functional domain (residue: 18-615) - IgG4
Fc protein sequence
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDOWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHYTQKSLSLSLGK
>Sequence ID 17: huACE2 functional domain (residue: 18-615) - IgA1
Fc Protein sequence
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDONKNSFVGWSTDWSPYADSQDVTVPCPVPSTPPTPSPSTPPTPSPSCCHPRLSLHRPALE
DLLLGSEANLTCTLTGLRDASGVTFTWTPSSGKSAVQGPPERDLCGCYSVSSVLPGCAEPWNHG
KTFTCTAAYPESKTPLTATLSKSGNTFRPEVHLLPPPSEELALNELVTLTCLARGFSPKDVLVR
WLQGSQELPREKYLTWASROEPSQGTTTFAVTSILRVAAEDWKKGDTFSCMVGHEALPLAFTQK
TIDRLAGKPTHVNVSVVMAEVDGTCY
>Sequence ID 18: huACE2 functional domain (residue: 18-615) - IgA2
Fc Protein sequence
QSTIEEQAKTFLDKFNHEAEDLFYQSSLASWNYNTNITEENVQNMNNAGDKWSAFLKEQSTLAQ
MYPLQEIQNLTVKLQLQALQQNGSSVLSEDKSKRLNTILNTMSTIYSTGKVCNPDNPQECLLLE
PGLNEIMANSLDYNERLWAWESWRSEVGKQLRPLYEEYVVLKNEMARANHYEDYGDYWRGDYEV
NGVDGYDYSRGQLIEDVEHTFEEIKPLYEHLHAYVRAKLMNAYPSYISPIGCLPAHLLGDMWGR
FWTNLYSLTVPFGQKPNIDVTDAMVDQAWDAQRIFKEAEKFFVSVGLPNMTQGFWENSMLTDPG
NVQKAVCHPTAWDLGKGDFRILMCTKVTMDDFLTAHHEMGHIQYDMAYAAQPFLLRNGANEGFH
EAVGEIMSLSAATPKHLKSIGLLSPDFQEDNETEINFLLKQALTIVGTLPFTYMLEKWRWMVFK
GEIPKDQWMKKWWEMKREIVGVVEPVPHDETYCDPASLFHVSNDYSFIRYYTRTLYQFQFQEAL
CQAAKHEGPLHKCDISNSTEAGQKLFNMLRLGKSEPWTLALENVVGAKNMNVRPLLNYFEPLFT
WLKDQNKNSFVGWSTDWSPYADSQDVTVPCRVPPPPPCCHPRLSLHRPALEDLLLGSEANLTCT
LTGLRDASGATFTWTPSSGKSAVQGPPERDLCGCYSVSSVLPGCAQPWNHGETFTCTAAHPELK
TPLTANITKSGNTFRPEVHLLPPPSEELALNELVTLTCLARGFSPKDVLVRWLQGSQELPREKY
LTWASRQEPSQGTTTYAVTSILRVAAEDWKKGETFSCMVGHEALPLAFTQKTIDRMAGKPTHIN
VSVVMAEADGTCY
>Sequence ID 19: SI-69R3_human ACE2-ECD-1-615-Fc-w2 (EU numbering
216-447) -protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDOAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDONKNSFVGWSTDWSPYADEPKSSDKTHTCPPCPAPELLGGPSV
FLFPPKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQQGNVFSCSVMHEAL
HNHYTQKSLSLSPGK
>Sequence ID 20: SI-69R3_human ACE2-ECD-1-615-Fc-w2-DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTCCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACGAGCCCAAATC
TTCCGACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACATGCG
TGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGAGAACCACA
GGTGTACACCCTGCCCCCATCCCGGGATGAGCTGACCAAGAACCAGGTCAGCCTGACCTGCCTG
GTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATAGCAAGCTCAC
CGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTAAATAG
>Sequence ID 21: SI-69R4-human ACE2-ECD-1-740(TMPRSS2 protease
cutting site) -Fc-w2 (EU numbering 216-447) -protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDOAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDQNKNSFVGWSTDWSPYADQSIKVRISLKSALGDKAYEWNDNEM
YLFRSSVAYAMRQYFLKVKNQMILFGEEDVRVANLKPRISFNFFVTAPKNVSDIIPRTEVEKAI
RMSRSRINDAFRLNDNSLEFLGIQPTLGPPNQPPVSEPKSSDKTHTCPPCPAPELLGGPSVFLF
PPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNH
YTQKSLSLSPGK
>Sequence ID 22: SI-69R4_human ACE2-ECD-1-740-Fc-w2-DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTCCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACCAAAGCATCAA
AGTGAGGATAAGCCTAAAATCAGCTCTTGGAGATAAAGCATATGAATGGAACGACAATGAAATG
TACCTGTTCCGATCATCTGTTGCATATGCTATGAGGCAGTACTTTTTAAAAGTAAAAAATCAGA
TGATTCTTTTTGGGGAGGAGGATGTGCGAGTGGCTAATTTGAAACCAAGAATCTCCTTTAATTT
CTTTGTCACTGCACCTAAAAATGTGTCTGATATCATTCCTAGAACTGAAGTTGAAAAGGCCATC
AGGATGTCCCGGAGCCGTATCAATGATGCTTTCCGTCTGAATGACAACAGCCTAGAGTTTCTGG
GGATACAGCCAACACTTGGACCTCCTAACCAGCCCCCTGTTTCCGAGCCCAAATCTTCCGACAA
AACTCACACATGCCCACCGTGCCCAGCACCTGAACTCCTGGGGGGACCGTCAGTCTTCCTCTTC
CCCCCAAAACCCAAGGACACCCTCATGATCTCCCGGACCCCTGAGGTCACATGCGTGGTGGTGG
ACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACCACAGGTGTACAC
CCTGCCCCCATCCCGGGATGAGCTGACCAAGAACCAGGTCAGCCTGACCTGCCTGGTCAAAGGC
TTCTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTATAGCAAGCTCACCGTGGACAA
GAGCAGGTGGCAGCAGGGGAACGTCTTCTCATGCTCCGTGATGCATGAGGCTCTGCACAACCAC
TACACGCAGAAGAGCCTCTCCCTGTCTCCGGGTAAATAG
>Sequence ID 23: SI-69R1_huACE2 functional domain (residue: 1-
615) - 6XHis protein sequence
MSSSSWLLLSLVAVTAAQSTIEEQAKTFLDKFNHEAEDLFYQSSLASWNYNTNITEENVQNMNN
AGDKWSAFLKEQSTLAQMYPLQEIQNLTVKLQLQALQQNGSSVLSEDKSKRLNTILNTMSTIYS
TGKVCNPDNPQECLLLEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLHAYVRAKLMNAYPSYI
SPIGCLPAHLLGDMWGRFWTNLYSLTVPFGQKPNIDVTDAMVDQAWDAQRIFKEAEKFFVSVGL
PNMTQGFWENSMLTDPGNVQKAVCHPTAWDLGKGDFRILMCTKVTMDDFLTAHHEMGHIQYDMA
YAAQPFLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPASLFHVSNDYSF
IRYYTRTLYQFQFQEALCQAAKHEGPLHKCDISNSTEAGQKLFNMLRLGKSEPWTLALENVVGA
KNMNVRPLLNYFEPLFTWLKDONKNS FVGWSTDWSPYADHHHHHH
>Sequence ID 24: SI-69R1_huACE2 functional domain (residue: 1-
615) - 6XHis DNA sequence
ATGTCAAGCTCTTCCTGGCTCCTTCTCAGCCTTGTTGCTGTAACTGCTGCTCAGTCCACCATTG
AGGAACAGGCCAAGACATTTTTGGACAAGTTTAACCACGAAGCCGAAGACCTGTTCTATCAAAG
TTCACTTGCTTCTTGGAATTATAACACCAATATTACTGAAGAGAATGTCCAAAACATGAATAAT
GCTGGGGACAAATGGTCTGCCTTTTTAAAGGAACAGTCCACACTTGCCCAAATGTATCCACTAC
AAGAAATTCAGAATCTCACAGTCAAGCTTCAGCTGCAGGCTCTTCAGCAAAATGGGTCTTCAGT
GCTCTCAGAAGACAAGAGCAAACGGTTGAACACAATTCTAAATACAATGAGCACCATCTACAGT
ACTGGAAAAGTTTGTAACCCAGATAATCCACAAGAATGCTTATTACTTGAACCAGGTTTGAATG
AAATAATGGCAAACAGTTTAGACTACAATGAGAGGCTCTGGGCTTGGGAAAGCTGGAGATCTGA
GGTCGGCAAGCAGCTGAGGCCATTATATGAAGAGTATGTGGTCTTGAAAAATGAGATGGCAAGA
GCAAATCATTATGAGGACTATGGGGATTATTGGAGAGGAGACTATGAAGTAAATGGGGTAGATG
GCTATGACTACAGCCGCGGCCAGTTGATTGAAGATGTGGAACATACCTTTGAAGAGATTAAACC
ATTATATGAACATCTTCATGCCTATGTGAGGGCAAAGTTGATGAATGCCTATCCTTCCTATATC
AGTCCAATTGGATGCCTCCCTGCTCATTTGCTTGGTGATATGTGGGGTAGATTTTGGACAAATC
TGTACTCTTTGACAGTTCCCTTTGGACAGAAACCAAACATAGATGTTACTGATGCAATGGTGGA
CCAGGCCTGGGATGCACAGAGAATATTCAAGGAGGCCGAGAAGTTCTTTGTATCTGTTGGTCTT
CCTAATATGACTCAAGGATTCTGGGAAAATTCCATGCTAACGGACCCAGGAAATGTTCAGAAAG
CAGTCTGCCATCCCACAGCTTGGGACCTGGGGAAGGGCGACTTCAGGATCCTTATGTGCACAAA
GGTGACAATGGACGACTTCCTGACAGCTCATCATGAGATGGGGCATATCCAGTATGATATGGCA
TATGCTGCACAACCTTTTCTGCTAAGAAATGGAGCTAATGAAGGATTCCATGAAGCTGTTGGGG
AAATCATGTCACTTTCTGCAGCCACACCTAAGCATTTAAAATCCATTGGTCTTCTGTCACCCGA
TTTTCAAGAAGACAATGAAACAGAAATAAACTTCCTGCTCAAACAAGCACTCACGATTGTTGGG
ACTCTGCCATTTACTTACATGTTAGAGAAGTGGAGGTGGATGGTCTTTAAAGGGGAAATTCCCA
AAGACCAGTGGATGAAAAAGTGGTGGGAGATGAAGCGAGAGATAGTTGGGGTGGTGGAACCTGT
GCCCCATGATGAAACATACTGTGACCCCGCATCTCTGTTCCATGTTTCTAATGATTACTCATTC
ATTCGATATTACACAAGGACCCTTTACCAATTCCAGTTTCAAGAAGCACTTTGTCAAGCAGCTA
AACATGAAGGCCCTCTGCACAAATGTGACATCTCAAACTCTACAGAAGCTGGACAGAAACTGTT
CAATATGCTGAGGCTTGGAAAATCAGAACCCTGGACCCTAGCATTGGAAAATGTTGTAGGAGCA
AAGAACATGAATGTAAGGCCACTGCTCAACTACTTTGAGCCCTTATTTACCTGGCTGAAAGACC
AGAACAAGAATTCTTTTGTGGGATGGAGTACCGACTGGAGTCCATATGCAGACCATCATCACCA
TCACCAC
>Sequence ID 25: SI-69R10_Human TMPRSS2 protein, His-tagged
(106-492) - protein sequence
MYRMQLLSCIALSLALVTNSWKFMGSKCSNSGIECDSSGTCINPSNWCDGVSHCPGGEDENRCV
RLYGPNFILQVYSSQRKSWHPVCQDDWNENYGRAACRDMGYKNNFYSSQGIVDDSGSTSFMKLN
TSAGNVDIYKKLYHSDACSSKAVVSLRCIACGVNLNSSRQSRIVGGESALPGAWPWQVSLHVQN
VHVCGGSIITPEWIVTAAHCVEKPLNNPWHWTAFAGILRQSFMFYGAGYQVEKVISHPNYDSKT
KNNDIALMKLQKPLTFNDLVKPVCLPNPGMMLQPEQLCWISGWGATEEKGKTSEVLNAAKVLLI
ETQRCNSRYVYDNLITPAMICAGFLQGNVDSCQGDSGGPLVTSKNNIWWLIGDTSWGSGCAKAY
RPGVYGNVMVFTDWIYRQMRADGHHHHHH
>Sequence ID 26: SI-69R10_Human TMPRSS2 protein, His-tagged
(106-492) - DNA sequence
ATGTACAGGATGCAACTCCTGTCTTGCATTGCACTAAGTCTTGCACTTGTCACCAATTCGTGGA
AGTTTATGGGTTCTAAATGCTCTAATAGCGGGATAGAATGTGACAGTAGTGGCACTTGCATTAA
CCCTTCAAACTGGTGTGATGGGGTAAGCCATTGCCCCGGGGGGGAAGATGAAAATAGATGTGTT
AGGCTCTACGGTCCCAACTTTATACTCCAGGTATATTCAAGTCAACGCAAATCATGGCATCCAG
TGTGTCAAGACGACTGGAACGAAAACTATGGACGCGCTGCATGTCGAGATATGGGATATAAGAA
TAACTTCTATAGTTCACAGGGAATCGTAGATGACTCTGGATCTACTAGTTTCATGAAACTGAAC
ACCTCTGCCGGAAACGTAGATATATATAAAAAGCTTTACCACTCCGACGCTTGTAGCTCTAAGG
CCGTAGTTAGCCTCAGATGCATCGCCTGCGGAGTAAACCTCAATTCATCTCGCCAGAGTAGGAT
CGTTGGCGGGGAAAGCGCCCTCCCAGGCGCTTGGCCTTGGCAAGTTTCCCTTCATGTCCAGAAT
GTTCATGTATGTGGCGGGTCTATAATCACCCCAGAATGGATCGTCACAGCTGCCCACTGCGTGG
AGAAACCCCTCAACAATCCTTGGCATTGGACCGCATTTGCCGGAATACTGAGACAATCATTTAT
GTTCTATGGAGCCGGGTACCAAGTCGAAAAGGTCATTTCCCATCCCAATTATGATTCCAAAACC
AAAAACAATGACATAGCCTTGATGAAACTCCAGAAGCCTTTGACATTTAATGACCTGGTCAAAC
CAGTGTGCCTCCCAAATCCTGGAATGATGTTGCAGCCTGAACAGTTGTGCTGGATCAGCGGTTG
GGGTGCTACCGAGGAGAAGGGTAAGACAAGCGAGGTCCTTAACGCTGCAAAGGTTTTGCTGATA
GAAACACAGAGATGTAACAGCCGCTATGTGTACGATAACCTGATCACCCCAGCTATGATTTGCG
CCGGGTTTTTGCAAGGTAACGTCGATTCTTGCCAAGGTGACTCAGGCGGCCCTCTTGTTACATC
AAAGAACAATATATGGTGGCTTATCGGCGATACATCATGGGGTTCTGGATGTGCTAAAGCCTAT
CGCCCAGGGGTGTATGGCAATGTAATGGTGTTTACAGACTGGATCTATAGGCAGATGCGGGCTG
ACGGTCACCATCATCACCATCACTGA
>Sequence ID 27: IgJ chain
MKNHLLFWGVLAVFIKAVHVKAQEDERIVLVDNKCKCARITSRIIRSSEDPNEDIVERNIRIIV
PLNNRENISDPTSPLRTRFVYHLSDLCKKCDPTEVELDNQIVTATQSNICDEDSATETCYTYDR
NKCYTAVVPLVYGGETKMVETALTPDACYPD
>Sequence ID 28: Secretory Component
KSPIFGPEEVNSVEGNSVSITCYYPPTSVNRHTRKYWCRQGARGGCITLISSEGYVSSKYAGRA
NLTNFPENGTFVVNIAQLSQDDSGRYKCGLGINSRGLSFDVSLEVSQGPGLLNDTKVYTVDLGR
TVTINCPFKTENAQKRKSLYKQIGLYPVLVIDSSGYVNPNYTGRIRLDIQGTGQLLFSVVINQL
RLSDAGQYLCQAGDDSNSNKKNADLQVLKPEPELVYEDLRGSVTFHCALGPEVANVAKFLCRQS
SGENCDVVVNTLGKRAPAFEGRILLNPQDKDGSFSVVITGLRKEDAGRYLCGAHSDGQLQEGSP
IQAWQLFVNEESTIPRSPTVVKGVAGGSVAVLCPYNRKESKSIKYWCLWEGAQNGRCPLLVDSE
GWVKAQYEGRLSLLEEPGNGTFTVILNQLTSRDAGFYWCLTNGDTLWRTTVEIKIIEGEPNLKV
PGNVTAVLGETLKVPCHFPCKFSSYEKYWCKWNNTGCQALPSQDEGPSKAFVNCDENSRLVSLT
LNLVTRADEGWYWCGVKQGHFYGETAAVYVAVEERKAAGSRDVSLAKADAAPDEKVLDSGFREI
ENKAIQDPR

What is claimed is:

1. A fusion protein, comprising a variant angiotensin converting enzyme 2 (ACE2) domain covalently fused to a Fc domain, wherein the variant ACE2 domain comprises a N-terminal deletion, a C-terminal deletion, or both, relative to a full-length wildtype ACE2 having a SEQ ID NO: 1, wherein the variant ACE2 domain has ACE2 activity.

2. (canceled)

3. The fusion protein of claim 1, wherein the variant ACE2 domain comprises an amino acid sequence of SEQ ID NO: 3.

4. (canceled)

5. The fusion protein of claim 1, wherein the Fc domain is derived from a Fc domain of an immunoglobulin, wherein the immunoglobulin is selected from IgG1, IgG2, IgG3, IgG4, IgA1 (d-IgA1, 5-IgA1), IgA2, IgD, IgE, or IgM.

6. (canceled)

7. The fusion protein of claim 1, wherein the Fc domain comprises a null mutation selected from K322A, L234A, and L235A compared to a wild type Fc domain having a SEQ ID NO: 5.

8. The fusion protein of claim 1, wherein the Fc domain comprises an amino acid sequence comprising SEQ ID NO: 6.

9. The fusion protein of claim 1, comprising an amino acid sequence selected from SEQ ID NO. 7, 9, 11, 13, 15, 16, 17, 18, 19, or 21.

10. The fusion protein of claim 1, wherein the Fc domain lacks effector function.

11-13. (canceled)

14. A fusion protein complex, comprising two fusion proteins of claim 1, wherein two fusion proteins are paired through a disulfide bond.

15-16. (canceled)

17. The fusion protein of claim 1, wherein the fusion protein has a binding affinity to SARS-CoV-2, SARS-CoV, or SARS spike protein with an equilibrium dissociation constant not greater than 50 nM.

18. A protein complex, comprising the fusion protein of claim 1 bound to a viral protein, wherein the viral protein comprises SARS-CoV-2, SARS-CoV, SARS spike protein, coronavirus, SARS virus, or a fragment or a combination thereof.

19. A protein complex, comprising the fusion complex of claim 14 bound to a viral protein, wherein the viral protein comprises SARS-CoV-2, SARS-CoV, SARS spike protein, coronavirus, SARS virus, or a fragment or a combination thereof.

20. An isolated nucleic acid encoding the fused protein of claim 3 comprising SEQ ID NO: 8.

21-23. (canceled)

24. A protein-conjugate, comprising the fusion protein complex of claim 14 and a drug moiety, wherein the drug moiety is linked to the fused protein through a linker, and wherein the linker comprises a covalent bond selected from an ester bond, an ether bond, an amine bond, an amide bond, a disulphide bond, an imide bond, a sulfone bond, a phosphate bond, a phosphorus ester bond, a peptide bond, a hydrazone bond or a combination thereof.

25. The protein-conjugate according to claim 24, wherein the drug moiety comprises an antiviral agent, an immune regulatory reagent, an imaging agent or a combination thereof.

26. The protein-conjugate according to claim 25, wherein the antiviral agent is selected from favipiravir, ribavirin, galidesivir, remdesvir, or a combination thereof.

27. The protein-conjugate according to claim 25, wherein the imaging agent may be radionuclide, a florescent agent, a quantum dots, or a combination thereof.

28. A pharmaceutical composition, comprising the fusion protein complex of claim 14 and a pharmaceutically acceptable carrier.

29. (canceled)

30. A pharmaceutical composition, comprising the protein-conjugate of claim 24 and a pharmaceutically acceptable carrier.

31. A method of treating or preventing a viral infection, acute respiratory distress syndrome, pulmonary arterial hypertension, or acute lung injury in a subject, comprising administering to the subject an effective amount of the fusion protein complex of claim 14.

32. The method of claim 31, further comprising co-administering an effective amount of a therapeutic agent, wherein the therapeutic agent comprises an antiviral agent.

33-37. (canceled)


SEQUENCE LISTING (28 sequences)

SEQ ID NO: 1 - protein, 805 aa, Artificial Sequence [sequence omitted]

SEQ ID NO: 2 - DNA, 2418 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 3 - protein, 615 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - DNA, 1845 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagac

SEQ ID NO: 5 - protein, 232 aa, Artificial Sequence
  Synthesized
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 232 aa, Artificial Sequence
  Synthesized
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 847 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 2544 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagcccaaatcttccgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 9 - protein, 844 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 2535 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagtccaaatatggtcccccgtgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctccaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaatga

SEQ ID NO: 11 - protein, 875 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 12 - DNA, 2628 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgcggcttcagccccaaggacgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctactga

SEQ ID NO: 13 - protein, 862 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 14 - DNA, 2589 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacagccaggatgtgactgtgccctgccgagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccacctatgctgtgaccagcatactgcgcgtggcagccgaggactggaagaagggggaaaccttctcctgcatggtgggccacgaggccctgccgctggccttcacacagaagaccatcgaccgcatggcgggtaaacccacccatatcaatgtgtctgttgtcatggcggaggcggacggcacctgctactga

SEQ ID NO: 15 - protein, 830 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 827 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 858 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 18 - protein, 845 aa, Artificial Sequence
  Synthesized
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 19 - protein, 847 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 2544 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagctccagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacgagcccaaatcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 21 - protein, 972 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 2919 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagctccagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccgagcccaaatcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 23 - protein, 621 aa, Artificial Sequence
  Synthesized
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADHHHHHH

SEQ ID NO: 24 - DNA, 1863 bp, Artificial Sequence
  Synthesized
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccatcatcaccatcaccac

SEQ ID NO: 25 - protein, 413 aa, Artificial Sequence
  Synthesized
MYRMQLLSCIALSLALVTNSWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADGHHHHHH

SEQ ID NO: 26 - DNA, 1242 bp, Artificial Sequence
  Synthesized
atgtacaggatgcaactcctgtcttgcattgcactaagtcttgcacttgtcaccaattcgtggaagtttatgggttctaaatgctctaatagcgggatagaatgtgacagtagtggcacttgcattaacccttcaaactggtgtgatggggtaagccattgccccgggggggaagatgaaaatagatgtgttaggctctacggtcccaactttatactccaggtatattcaagtcaacgcaaatcatggcatccagtgtgtcaagacgactggaacgaaaactatggacgcgctgcatgtcgagatatgggatataagaataacttctatagttcacagggaatcgtagatgactctggatctactagtttcatgaaactgaacacctctgccggaaacgtagatatatataaaaagctttaccactccgacgcttgtagctctaaggccgtagttagcctcagatgcatcgcctgcggagtaaacctcaattcatctcgccagagtaggatcgttggcggggaaagcgccctcccaggcgcttggccttggcaagtttcccttcatgtccagaatgttcatgtatgtggcgggtctataatcaccccagaatggatcgtcacagctgcccactgcgtggagaaacccctcaacaatccttggcattggaccgcatttgccggaatactgagacaatcatttatgttctatggagccgggtaccaagtcgaaaaggtcatttcccatcccaattatgattccaaaaccaaaaacaatgacatagccttgatgaaactccagaagcctttgacatttaatgacctggtcaaaccagtgtgcctcccaaatcctggaatgatgttgcagcctgaacagttgtgctggatcagcggttggggtgctaccgaggagaagggtaagacaagcgaggtccttaacgctgcaaaggttttgctgatagaaacacagagatgtaacagccgctatgtgtacgataacctgatcaccccagctatgatttgcgccgggtttttgcaaggtaacgtcgattcttgccaaggtgactcaggcggccctcttgttacatcaaagaacaatatatggtggcttatcggcgatacatcatggggttctggatgtgctaaagcctatcgcccaggggtgtatggcaatgtaatggtgtttacagactggatctataggcagatgcgggctgacggtcaccatcatcaccatcactga

SEQ ID NO: 27 - protein, 159 aa, Artificial Sequence
  Synthesized
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 28 - protein, 585 aa, Artificial Sequence
  Synthesized
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR